(19) United States
(12) Patent Application Publication
(10) Pub. No.: US 2009/0197851 A1
(43) Pub. Date: Aug. 6, 2009

(54) THERAPY AND USE OF COMPOUNDS IN THERAPY

(75) Inventors: Stefan Anker, Berlin (DE); Andrew Coats, London (GB); Hans-Dieter Volk, Berlin (DE); Mathias Rauchhaus, Halle (DE); Ralf Reiner Schumann, Zepernick (DE)

Correspondence Address: NORRIS, MCLAUGHLIN & MARCUS, P.A., 875 THIRD AVE, 18TH FLOOR, NEW YORK, NY, 10022, US

(73) Assignee: MAX-DELBRUCK-CENTRUM FUR MOLEKULARE MEDIZIN, Berlin (DE)

(21) Appl. No.: 12/187,169

(22) Filed: Aug. 6, 2008

Related U.S. Application Data
(63) Continuation of application No. 09/980,645, filed on Mar. 20, 2002, now abandoned.

(30) Foreign Application Priority Data
Mar. 9, 1999 (GB) 9905300.1
Mar. 9, 1999 (GB) 9905307.6
Mar. 9, 1999 (GB) 9905310.0
Mar. 9, 1999 (GB) 9905314.2
Mar. 9, 1999 (GB) 9905315.9

(51) Int. Cl. A61K 31/575; A61P 9/00

(52) U.S. Cl. 514/182

(57) ABSTRACT

A method of treating, preventing or ameliorating chronic heart failure or acute heart failure in a patient the method comprising administering to the patient an effective amount of: a compound that is able to bind to an endotoxin (lipopolysaccharide; LPS) molecule, for example LPS binding protein, BPI, lipoproteins, bile acids or an antibody capable of binding LPS, a compound that is able to bind to an endotoxin (lipopolysaccharide; LPS) molecule or bacterium in the gut, for example charcoal, a bile acid or Fuller's earth, an antibacterial agent that is substantially active in the gut, an agent that is able to inhibit the response by a cell to endotoxin (lipopolysaccharide; LPS), an agent that may form a barrier or that otherwise impedes translocation of bacteria or endotoxin (LPS) from the gut into the patient's circulation. A method of treating, preventing or ameliorating endotoxin-mediated immune activation in acute or chronic heart failure in a patient the method comprising administering to the patient an effective amount of: a compound that is able to bind to an endotoxin (lipopolysaccharide; LPS) molecule, for example LPS binding protein, BPI, lipoproteins, bile acids or an antibody capable of binding LPS, a compound that is able to bind to an endotoxin (lipopolysaccharide; LPS) molecule or bacterium in the gut, for example charcoal, a bile acid or Fuller's earth, an antibacterial agent that is substantially active in the gut, an agent that is able to inhibit the response by a cell to endotoxin (lipopolysaccharide; LPS), an agent that may form a barrier or that otherwise impedes translocation of bacteria or endotoxin (LPS) from the gut into the patient's circulation.

The present invention relates to therapy and the use of compounds in therapy. In particular, it relates to the treatment and prevention of endotoxin-mediated immune activation in acute and chronic heart failure (CHF). The present invention also relates to therapy and the use of agents in the therapy of cachexia and wasting syndromes due to diseases other than congestive heart failure.

Chronic heart failure is a heterogeneous syndrome with an overall adverse prognosis. It is a disease in which there is a failure to pump enough blood around the body to meet its needs. Two particular predictors of adverse prognosis are neurohormonal abnormalities (Packer (1992) J Am Coll Cardiol 20, 248-254) and the development of cachexia (Abel et al (1976) Arch Surg 111, 45-50).

The syndrome of cardiac cachexia has been recognized for many centuries (Katz et al (1962) Br Heart J 24, 257-264), but little is known about the mechanisms of the transition from heart failure to cardiac cachexia. Even the definition of cachexia and the characteristics of the cachectic patient are controversial. More than 30 years ago, the pathogenesis of cardiac cachexia was linked to dietary and metabolic factors (Pittman & Cohen (1964) New Eng J Med 271, 403-409). In 1990, Levine et al ((1990) New Eng J Med 323, 236-241) and subsequently others (McMurray et al (1991) Br Heart J 66, 356-358; Dutka et al (1993) Br Heart J 70, 141-143) showed the TNF-α in plasma is increased in patients with severe heart failure and coexisting cardiac cachexia, as in other wasting disorders. The plasma concentrations of TNF-α partly reflect the local tissue concentration, which is more closely related to muscle wasting (Tracey et al (1990) J Clin Invest 86, 2014-2024). Cytokine activation is a potential causal mechanism for the development of cachexia.

Cardiac cachectic patients suffer from loss of both muscle (ie protein reserves) and fat tissue (ie energy reserves), indicative of increased catabolism. An increased resting metabolic rate, regulated primarily by thyroid hormones (Himms-Hagen et al (1993) In: Grandier R. Stock, eds, Mammalian Thermogenesis, Chapman & Hall, London, UK) and catecholamines (Poehlman & Danforth (1991) Am J Physiol 261, E233-E239), has been reported in CHF patients (Poehlman et al (1994) Ann Intern Med 121, 860-862). Cortisol, another catabolic hormone, is also increased in untreated severe congested heart failure patients (Anand et al (1989) Circulation 80, 299-305). Less is known about anabolic metabolism in heart failure. Anand et al ((1989) Circulation 80, 299-305) found hGH to be greatly increased (≈10-fold) in untreated patients with severe heart failure. To date, these results have not been confirmed by others. Increased plasma insulin levels and insulin resistance occur in patients with CHF (Swan et al (1994) Eur Heart J 15, 1528-1532).

The neurohormonal hypothesis (Packer (1992) J Am Coll Cardiol 20, 248-254) postulates that heart failure progresses because activated endogenous neurohormonal systems exert a deleterious effect on the heart and circulation. Several studies have found neurohormonal activation to be strongly related to mortality (Cohn et al (1984) New Eng J Med 311, 819-823; Swedberg et al (1990) Circulation 82, 1730-1736; Francis et al (1993) Circulation 87, (Suppl VI) VI-40-VI-48) but different hormones correlate only weakly with each other (Swedberg et al (1990) Circulation 82, 1730-1736). Norepinephrine and plasma renin activity were found not to be related to peak oxygen consumption (peak VO₂) or LVEF (Francis et al (1993) Circulation 87, (Suppl VI) VI-40-VI-48). Left ventricular function, exercise capacity, clinical status, and sympathetic activation were independently related to the progression of CHF (Francis et al (1993) Circulation 87, (Suppl VI) VI-40-VI-48).

Anker et al (1997) Circulation 96, 526-534 describes a study of the hormonal changes and catabolic/anabolic imbalance in CHF and concludes that cachexia is more closely associated with hormonal changes in CHF than conventional measures of the severity of CHF and suggests that the syndrome of heart failure progresses to cardiac cachexia if the normal metabolic balance between catabolism and anabolism is altered.

Anker et al (1997) The Lancet 349, 1050-1053 suggests that the cachectic state is a strong independent risk factor for mortality in patients with CHF.

Anker et al (1997) J Am Coll Cardiol 30, 997-1001 describes investigations of tumour necrosis factor (TNF) and steroid metabolism is CHF and concludes that there is an increase in TNF and its soluble receptor in CHF and that this increase is associated with a rise in the cortisol/DHEA (catabolic/anabolic) ratio. These changes correlate with body mass index and clinical severity of heart failure, suggesting a possible etiological link.

Anker et al (1997) Am J Cardiol 79, 1426-1430 suggests that a chronic endotoxin challenge may cause immune activation in CHF and indicates that patients with high soluble CD14 levels have markedly increased levels of TNF-α, soluble TNF receptors 1 and 2, and intracellular adhesion molecule-1.

Starr et al (1995) Direct action of endotoxin on cardiac muscle Shock 3(5), 380-384 suggest that endotoxin directly affects the contractile response of cardiac muscle to calcium. Endotoxin is known to be the strongest biological stimulus for cytokine production, in particular for production of TNFα. A variety of pathophysiologic processes that directly or indirectly could contribute to deterioration of heart failure are influenced by immune activation, and specifically by TNFα

a) TNF is detrimental for endothelial function and peripheral blood flow. In the short term TNF can up-regulate iNOS (as is seen in sepsis) and thereby contribute to vasodilation, but chronically TNF may in particular down-regulate cNOS. We have found a strong inverse correlation between the levels of TNF and the peak leg blood flow response to ischaemia (r=−0.7, p<0.0001). Impaired peripheral blood flow is closely linked to exercise capacity in CHF patients—particularly in cachectic patients.
b) Impaired peripheral blood flow is also an important component of the insulin resistance syndrome that we have shown to be present in CHF-insulin resistance appears to be a cause of energy depletion in the peripheral musculature.
c) TNF has negative inotropic effects on the heart (Starr et al (1995) Shock 3(5), 380-384.
d) The immune activation status in CHF is closely linked to the hormonal catabolic/anabolic balance in CHF patients (Anker et al (1997) J Am Coll Cardiol 30, 997-1001).
e) TNF is the strongest correlate of the degree of weight loss in cachectic CHF patients.
f) TNF could trigger cell apoptosis—not only in the heart, but particularly also in the periphery. This could lead to tissue dysfunction, and finally to specific and/or general tissue wasting. General wasting is then closely related to impaired prognosis in CHF.

The principal primary natural bile acids, cholic acid and chenodeoxycholic acid, are produced in the liver from cholesterol and are conjugated with glycine and taurine to give glycocholic acid, taurocholic acid, glycochenodeoxycholic acid and taurochenodeoxycholic acid before being secreted into the bile where they are present as the sodium or potassium salts (bile salts). Secondary, natural bile acids are formed in the colon by bacterial deconjugation and 7-dehydroxylation of cholic acid and chenodeoxycholic acid producing deoxycholic acid and lithocholic acid, respectively. Ursodeoxycholic acid is a minor bile acid in man although it is the principal bile acid in bears. Dehydrocholic acid is a semi-synthetic bile acid.

The total body pool of bile salts is about 3 g, and most of the secreted bile salts are reabsorbed in a process of enterohepatic recycling, so that only a small fraction of this amount must be synthesised de novo each day. Bile salts are strongly amphiphilic; with the acid of phospholipids they form micelles and emulsify cholesterol and other lipids in bile. Oral administration of chemodeoxycholic acid also reduces the synthesis of cholesterol in the liver, while ursodeoxycholic acid reduces biliary cholesterol secretion apparently by increasing conversion of cholesterol to other bile acids. The bile acids (but not the bile salts) also have a choleretic action, increasing the secretion of bile, when given by mouth.

Chenodeoxycholic acid and ursodeoxycholic acid are given by mouth in the management of cholesterol-rich gallstones in patients unsuited to, or unwilling to undergo, surgery. Preparations containing bile salts have been used to assist the emulsification of fats and absorption of fat-soluble vitamins in conditions in which there is a deficiency of bile in the gastro-intestinal tract. Ox bile has also been used in the treatment of chronic constipation.

LPS binding protein is a serum protein which binds to LPS (Schumann et al (1990) Structure and function of lipopolysaccharide binding protein Science 249, 1429-1431). The ratio of LPS to LBP may affect the immunostimulatory effects of LPS (Tobias et al (1997) Lipopolysaccharide binding proteins BPI and LBP form different types of complexes with LPS J Biol Chem 272, 18682-18685), and the level of LBP in vivo can vary substantially due to transcriptional control of LBP production (Schumann et al (1996) Lipopolysaccharide binding protein (LBP) is a secretory class 1 acute phase protein requiring binding of the transcription factor STAT-3, C/EBPβ and AP-1 Mol Cell Biol 16, 3490-3503). High concentrations of LBP may completely block LPS effects in vitro and in a murine sepsis model (Lamping et al (1998) LPS-binding protein protects mice from septic shock caused by LPS or gram-negative bacteria J Clin Invest 101, 2065-2071).

Bactericidal/permeability-increasing protein (BPI) is a protein found in human white blood cells that has multiple anti-infective and binding properties. It is capable of killing bacteria, of enhancing the effectiveness of antibiotics and of binding to and neutralising endotoxin (lipopolysaccharide; LPS). A BPI-derived pharmaceutical preparation undergoing trial is Neuprex® (Xoma Corp).

Endotoxin (lipopolysaccharide; LPS) signalling may be mediated through the interaction of the CD14 molecule and toll-like receptor, particularly toll-like receptor 4 and 2, as discussed, for example, in Anker et al (1997) Am J Cardiol 79, 1426-1430, Wright (1991) Multiple receptors for endotoxin Curr Opin Immunol 3, 83-90 and Ulevitch & Tobias (1995) Receptor-dependent mechanisms of cell stimulation by bacterial endotoxin Ann Rev Immunol 13, 437 457, and Kirschning et al (1998), Human toll-like receptor 2 confers responsiveness to bacterial lipopolysaccharide. J Exp Med 188:2091-2097, and Chow et al (1999), Toll-like receptor-4 mediates lipopolysaccharide-induced signal transduction. J Biol Chem 274:10689-10692.

No one has previously proposed that:
a compound that is able to bind to an endotoxin (lipopolysaccharide; LPS) molecule, for example LPS binding protein, BPI, lipoproteins, bile acids or an antibody capable of binding LPS,
a compound that is able to bind to an endotoxin (lipopolysaccharide; LPS) molecule or bacterium in the gut, for example charcoal, a bile acid or Fuller's earth,
an antibacterial agent that is substantially active in the gut,
an agent that is able to inhibit the response by a cell to endotoxin (lipopolysaccharide; LPS),
an agent that may form a barrier or that otherwise impedes translocation of bacteria or endotoxin (LPS) from the gut into the patient's circulation
   be useful in the management of patients with either acute or chronic heart failure.

Through multiple pathways immune activation is detrimental for heart failure. We show here that endotoxin is raised in oedematous compared to non-oedematous heart failure, and propose that:
preventing or counteracting the presence of endotoxin or inhibiting its biological effects,
reducing the availability of LPS for absorption in the gut,
reducing the quantity of bacteria and hence endotoxin (LPS) in the gut,
inhibiting the response by cells to endotoxin (lipopolysaccharide; LPS),
reducing or blocking the permeability of the gut wall to bacteria and/or endotoxin (LPS)
   may lead to improved immune status, which could through multiple mechanisms improve the prognosis and clinical status of patients in the short and long term.

A first aspect of the invention provides a method of treating, preventing or ameliorating chronic heart failure or acute heart failure in a patient the method comprising administering to the patient an effective amount of a compound that is able to bind to an endotoxin (lipopolysaccharide; LPS) molecule, a compound that is able to bind to an endotoxin (lipopolysaccharide; LPS) molecule in the gut of the patient, an antibacterial agent (it is preferred that the antibacterial agent is active in the gut), a compound that is able to inhibit the response by a cell to endotoxin (LPS) and/or an agent that is able to reduce or substantially block the permeability of the gut wall to bacteria and/or endotoxin (LPS).

A second aspect of the invention provides a method of treating, preventing or ameliorating endotoxin-mediated immune activation in acute or chronic heart failure in a patient the method comprising administering to the patient an effective amount of a compound that is able to bind to an endotoxin (lipopolysaccharide; LPS) molecule, a compound that is able to bind to an endotoxin (lipopolysaccharide; LPS) molecule in the gut of the patient, an antibacterial agent (it is preferred that the antibacterial agent is active in the gut), a compound that is able to inhibit the response by a cell to endotoxin (LPS) and/or an agent that is able to reduce or substantially block the permeability of the gut wall to bacteria and/or endotoxin (LPS).

The following classes of patients in particular may benefit from treatment

1. Patients with acute heart failure (decompensated chronic heart failure, myocardial infarction).
2. Any decompensated heart failure patients with evidence of peripheral oedema.
3. Patients with severe heart failure (NYHA class III or IV) or with cardiac cachexia.
4. Stable CHF patients if any deterioration occurs, for example patients with a history of decompensation phases.

It is preferred that the patient has peripheral and/or bowel oedema.

Typically, in relation to the treatment of acute heart failure, the compound may be administered following myocardial infarction.

Acute heart failure is most frequently characterised by the presence of shortness of breath and oedema. It is most frequently treated by adjusting diuretics. It will be appreciated that the methods of the invention may be used in conjunction with other treatments for acute or chronic heart failure, for example treatment with diuretics. Thus, a further aspect of the invention is a method or use of the invention (as described below) wherein a diuretic is administered to the patient. The diuretic may be administered to the patient before, after or concurrently with the compound of the method or use of the invention.

It is preferred that the compound is able to substantially reduce the biological activity of endotoxin (lipopolysaccharide) such that the endotoxin has a substantially reduced effect on the liver or does not reach the liver in a substantially active form.

The compound may be, for example, a bile acid, a lipoprotein like for instance low density lipoprotein (LDL), high density lipoprotein (HDL), very low density lipoprotein (VLDL), apolipoprotein (a), or a lipoprotein mixture, BPI, LPS binding protein or a functional equivalent thereof or an antibody (which term includes an antibody fragment, as known to those skilled in the art) capable of binding to LPS. It will be appreciated that it is preferred that the compound is able to enter the circulation, for example following oral administration or inhalation, and is able to bind endotoxin (lipopolysaccharide; LPS) under physiological conditions in the circulation and/or tissues of the body, for example in the blood. The ability of a compound to bind LPS may be determined as known in the art, for example using methods set out in Schumann et al (1990) Science 249, 1429-1431.

A further aspect of the invention relates to the use of lipoproteins to bind LPS and to inhibit its biological activity. Lipoproteins could be, for instance but not exclusively, low density lipoprotein (LDL), high density lipoprotein (HDL), very low density lipoprotein (VLDL), apolipoprotein (a), or a lipoprotein mixture. It has never been proposed that the application of lipoproteins in patients with acute or chronic heart failure could be beneficial in general, that it could be of anti-inflammatory value, and that it could act in to prevent or treat cachexia. Current treatment guidelines suggest to lower lipoprotein levels in patients with heart failure and coronary artery disease.

We now show that high lipoprotein levels are related to better prognosis in CHF patients, and that low lipoprotein levels are related to impaired survival (example 3). We also show that lipoproteins inhibit cytokine production in vitro (example 4 and 5), and that higher plasma lipoprotein levels of patients with chronic heart failure and healthy subjects are related to less LPS-mediated cytokine production in whole blood tests in vitro (example 6).

A further aspect of the invention relates to the use of lipoproteins in combination with LPS-binding protein (LBP). We demonstrate that high levels of LBP can inhibit LPS-stimulated TNF production in lipoprotein free conditions (example 7) as well as in the presence of lipoproteins in serum (example 8), but complete inhibition of LPS-stimulated TNF production can best be achieved when both LBP and lipoproteins are present (example 7).

A further aspect of the invention is to use only those 3-hydroxy-3-methylglutaryl-coenzyme

A (HMG-CoA) reductase inhibitors for the treatment of patients with acute and chronic heart failure that are able to increase lipoprotein fractions (HDL, LDL, VLDL, or apolipoprotein (a)) and that at the same time do not lower LDL and/or cholesterol levels.

Lipid-lowering therapy with 3-hydroxy-3-methylglutaryl-coenzyme A (HMG-CoA) reductase inhibitors, referred to as the statins, have been shown to reduce morbidity and mortality in the primary and secondary prevention of coronary artery disease [Shepherd et al., N Engl J. Med. 1995; 333:1301-1307, Pedersen et al., Circulation. 1998; 97:1453-1460]. The drugs of this class that finally were chosen to be tested in clinical trials (for instance: simvastatin, fluvastatin, pravastatin, cerivastatin, lovastatin, atorvastin) were selected for their ability to lower LDL and cholesterol and it is known that they can increase HDL plasma levels. We now show that LDL and VLDL are particularly able to lower LPS-mediated cytokine production (example 6). For patients with heart failure benefits of the use of statins has not been documented, but it is commonly thought that such drugs should be used when cholesterol or LDL levels are high and coronary artery disease aetiology is suspected. Therefore, the use of studies has been recommended in recent heart failure treatment guidelines. We propose for the first time that reductions of lipoproteins and cholesterol in general and of LDL and VLDL in particular are not desirable in patients with acute and chronic heart failure. Therefore, the use of 3-hydroxy-3-methylglutaryl-coenzyme A reductase inhibitors that are able to increase lipoprotein fractions (HDL, LDL, VLDL, or apolipoprotein (a)) and that at the same time do not lower LDL and/or cholesterol levels would be beneficial in acute and chronic heart failure. The evidence not to lower LDL and cholesterol would come from randomised placebo-controlled studies of at least 6 months duration.

It will be appreciated that the blood of the patient may be exposed to the said compound outside the patient's body. Thus, haemoperfusion (the passage of blood through an absorbent material) may be useful in removing LPS from blood. The blood is returned to the patient after it has been passed through the absorbent material. The absorbent material may be, for example, activated charcoal or a synthetic hydrophobic polystyrene resins that is capable of binding to endotoxin, or is capable of binding a compound as described above that is capable of binding endotoxin.

It is preferred that the compound is able to substantially reduce the availability of endotoxin (lipopolysaccharide) for absorption from the gut, such that the amount of endotoxin that is absorbed is reduced or is less biologically active. Thus, the compound may promote the excretion of LPS.

It will be appreciated that the compound may bind to LPS or may bind to a bacterium that may comprise LPS.

The compound may be, for example, activated charcoal, a bile acid, Fuller's earth, attapulgite, kaolin or bentonite or a clay. It will be appreciated that it is preferred that the compound is able to bind LPS under physiological conditions in the gut. The ability of a compound to bind LPS may be determined using methods well known to those skilled in the art, for example making use of methods of quantifying LPS as described in Example 1.

It is preferred that the antibacterial agent is able to substantially reduce the amount of bacteria and/or free endotoxin (lipopolysaccharide) in the gut, such that the amount of endotoxin that is available to be absorbed is reduced. It is preferred that the antibacterial agent is a bactericidal agent.

It is preferred that the antibacterial agent is largely unabsorbed from the gut. Suitable antibacterial agents will be known to those skilled in the art. In general, aminoglcoside bactericidal antibiotics are poorly absorbed from the gut and may be particularly suitable. Examples include neomycin, framycetin, gentamycin, streptomycin and kanamycin. Some cephalosporin (cephem) antibiotics may also be useful. Cephalothin or cephazolin, for example, are poorly absorbed from the gut and have some activity against gram-negative bacteria. Cefotaxime, cefinenoxime, cefodizime, ceftizoxime and cetriaxone may also be suitable. Vancomycin hydrochloride (a glycopeptide) or the related teicoplanin may also be useful as they are poorly absorbed when taken by mouth. Bactericidal/permeability increasing protein (BPI) may act as an antibacterial agent. It may also enhance the effectiveness of other antibacterial agents. It is described, for example, in Beamer et al (1999) The three-dimensional structure of human bactericidal/permeability-increasing protein: implications for understanding protein-lipopolysaccharide interactions Biochem Pharmacol 57(3), 225-9.

It will be appreciated that the antibacterial agent administered to the patient may be a single chemical species, or it may be a mixture of two or more chemical species. Thus, for example, BPI may be administered with another antibacterial agent, for example neomycin.

The antibacterial agent may be administered to the patient in any suitable form or in any suitable way. The compound or a formulation thereof may be administered by any conventional method including oral or rectal administration. The treatment may consist of a single dose or a plurality of doses over a period of time.

Chronic intermittent use (for example, once or twice per year) may be particularly useful in order to reduce or prevent bacterial overgrowth of the gut and thereby reduce the potential for endotoxin or bacteria being absorbed from the gut.

The compound may decrease the endotoxin (LPS) sensitivity of, for example, immune system cells and thereby decrease the cytokine production by these cells, for example it may decrease the production of TNFα. It is preferred that the compound acts directly on a cell that is stimulated directly by endotoxin. It is further preferred that the compound acts to modulate signalling within a cell caused by endotoxin binding to or otherwise interacting with that cell. The agent may be IGF-1 or allopurinol, oxipurinol, or any other unspecific xanthine oxidase inhibitor, or a specific xanthine oxidase inhibitor (like TMX-67 of TAP Holdings Inc./USA). These compounds may decrease gut wall permeability, for example permeability to bacteria and/or endotoxin (lipopolysaccharide; LPS), by effects on the cells of the gut wall. Liquorice and its derivatives, for example carbenoxolone, may stimulate the synthesis of protective mucus which may also reduce the permeability of the gut wall to bacteria and/or endotoxin (LPS).

The agent may form a coating of the gut wall which may reduce or substantially block the permeability of the coated gut wall to bacteria and/or endotoxin (LPS). Thus, the coating may reduce the ease with which bacteria and/or endotoxin (LPS) may translocate from the gut to the patient's circulation. Alginates, for example, may form a gel over the gut surface and may therefore be useful. Also colostrum of human, bovine, or other mamallian origin, may be used to prevent uptake of endotoxin (LPS) from the gut into the circulation.

An enteric coated formulation, as know to those skilled in the art, may be useful in delivering the agent to the lower gastrointestinal tract, in particular the bowel.

Sulfacrate may coat the gastric mucosa (preferentially at sites of ulceration) by forming an adherent complex with proteins and may therefore be useful.

The agent may form a hydrogel. The hydrogel may be noninflammatory and biodegradable and may reduce the permeability of the gut wall to translocation of bacteria and/or endotoxin (LPS). Many such materials now are known, including those made from natural and synthetic polymers. In a preferred embodiment, the method exploits a hydrogel which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10 to about 80% by weight ethylene oxide and from about 20 to about 90% by weight propylene oxide. A particularly preferred hydrogel contains about 70% polyethylene oxide and 30% polypropylene oxide. Hydrogels which can be used are available, for example, from BASF Corp., Parsippany, N.J., under the tradename Pluronic®.

In this embodiment, the hydrogel is cooled to a liquid state and the oligonucleotides are admixed into the liquid to a concentration of about 1 mg oligonucleotide per gram of hydrogel. The resulting mixture then is applied onto the surface to be treated, for example by spraying or painting during surgery or using a catheter or endoscopic procedures. As the polymer warms, it solidifies to form a gel.

It is preferred that the agent is able to substantially reduce the amount of bacteria and/or free endotoxin (lipopolysaccharide) that is able to translocate from the gut into the circulation of the patient, such that the amount of endotoxin that is present in the circulation or tissues of the patient is reduced. Thus, the agent may reduce the amount of bacteria and/or free endotoxin (lipopolysaccharide) that is able to translocate from the gut into the circulation of the patient by about 30%, 50%, 80%, 90% or 99%. It is preferred that the agent is largely unabsorbed from the gut.

The agent may form a structure that resembles an sleeve or tube on the inside of the gut wall. Thus, structure may act as a “gut condom”. The structure may form a semi-permeable or substantially impermeable barrier between the portion of the gut where the structure is present and the circulation of the patient.

A further aspect of the invention provides a method of treating, preventing or ameliorating chronic heart failure or acute heart failure in a patient the method comprising administering to the patient an effective amount of:
a bile acid, BPI, a lipoprotein, LPS binding protein or a functional equivalent thereof or an antibody capable of binding to endotoxin,
activated charcoal, Fuller's earth, attapulgite, kaolin or bentonite or a clay,
an antibody able to bind the CD14 receptor, soluble CD14 receptor, or drug blocking effectively signalling through toll-like receptors, particularly toll-like receptor 4 and 2
IGF-1, allopurinol, oxipurinol, or any other unspecific xanthine oxidase inhibitor, or a specific xanthine oxidase inhibitor, liquorice or its derivatives, for example carbenoxolone, an alginate, sulfacrate, colostrum of human, bovine, or other mamallian origin or an agent that may form a hydrogel.

A still further aspect of the invention provides a method of treating, preventing or ameliorating endotoxin-mediated immune activation in acute or chronic heart failure in a patient the method comprising administering to the patient an effective amount of:
a bile acid, BPI, a lipoprotein, LPS binding protein or a functional equivalent thereof or an antibody capable of binding to endotoxin,
activated charcoal, Fuller's earth, attapulgite, kaolin or bentonite or a clay,
an antibody able to bind the CD14 receptor, soluble CD14 receptor, or drug blocking effectively signalling through toll-like receptors, particularly toll-like receptor 4 and 2
IGF-1, allopurinol, oxipurinol, or any other unspecific xanthine oxidase inhibitor, or a specific xanthine oxidase inhibitor, liquorice or its derivatives, for example carbenoxolone, an alginate, sulfacrate, colostrum of human, bovine, or other mamallian origin or an agent that may form a hydrogel.

By “bile acid” wc include all naturally occurring bile acids whether from man or from another animal. Also is included bile acids which are synthetic or semi-synthetic derivatives of naturally occurring bile acids. Of course, all bile acids including those that are “naturally occurring” may be synthesised chemically.

Bile acids are available from Falk Pharma GmbH and are described, for example, in WP96/17859, DE29717252 and WO98/05339.

Bile acids for use in the method of the invention include, but are not limited to, chemodeoxycholic acid (3α,7α-dihydroxy-5θ-cholan-24-oic acid), arsodeoxycholic acid (3α,7θ-dihydroxy-5θ-cholan-24-oic acid), dehydrocholic acid (3,7,12-trioxo-5θ-cholan-24-oic acid), cholic acid and deoxycholic acid.

Preferably, the bile acid is a bile acid which is able to form micelles. Preferably, the bile acid is able to form a micelle around an endotoxin (lipopolysacharide molecule). It is particularly preferred that the bile acid is able to bind to endotoxin (lipopolysaccharide) molecules and substantially reduce the available endotoxin in the patient. In particular, it is preferred if the bile acid is able to substantially reduce the biological activity of endotoxin (lipopolysaccharide) such that the endotoxin has a substantially reduced effect on the liver or does not reach the liver in a substantially active form.

It is preferred if the bile acid is any one of ursodeoxycholic acid, chemodeoxycholic acid, dehydrocholic acid, cholic acid and deoxycholic acid.

It is preferred if the bile acid is ursodeoxycholic acid.

By “LPS binding protein” is included the protein which binds to LPS (endotoxin) described in Schumann et al (1990) Structure and function of lipopolysaccharide binding protein Science 249, 1429-1431 and fragments, variants, fusions or derivatives thereof that are capable of binding to LPS, for example as determined in Schumann et al (1990). Further proteins that are capable of binding to LPS are known, for example as described in U.S. Pat. No. 5,760,177, isolated from horseshoe crab.

Bactericidal/permeability increasing protein (BPI) is described, for example, in Beamer et al (1999) The three-dimensional structure of human bactericidal/permeability-increasing protein: implications for understanding protein-lipopolysaccharide interactions Biochem Pharmacol 57(3), 225-9.

Antibodies that are capable of binding to endotoxin are well known to those skilled in the art, for example as described in U.S. Pat. No. 5,179,018 (Mammalian monoclonal antibodies against endotoxin of gram-negative bacteria) and U.S. Pat. No. 5,858,728 (Monoclonal antibody against LPS core).

The term “activated carbon” is well known in the art and includes material prepared from vegetable matter by carbonisation processes intended to confer a high absorbing power (BP form) or prepared by the destructive distillation of various organic materials, treated to increase its absorptive power (USP form). The BP form may adsorb not less than 40% of its own weight of phenazone, calculated with reference to the dried substance.

Fuller's earth consists largely of montmorillonite, a native hydrated aluminium silicate, with which very finely divided calcite (calcium carbonate) may be associated.

Preferably, the compound is able to bind to endotoxin (lipopolysaccharide) molecules and substantially reduce the absorbable endotoxin in the gut of the patient. The compound may promote excretion of the endotoxin.

The compound may act to reduce the level of receptors through which endotoxin (LPS) acts, for example CD14 receptors, for example by reducing the formation of receptors, for example CD14 receptors. Thus, the compound may interfere with the transcription or translation of the gene encoding the CD14 receptor. It may be an antisense compound, for example directed against the mRNA encoding the CD14 receptor. The CD14 receptor sequence is reported in, for example, Ferrero E & Goyert S M (1988) Nucleotide sequence of the gene encoding the monocyte differentiation antigen, CD14. Nucleic Acids Res 16(9), 4173. Thus, the compound may inhibit signalling via the CD14 receptor.

Antisense oligonucleotides are single-stranded nucleic acid, which can specifically bind to a complementary nucleic acid sequence. By binding to the appropriate target sequence, an RNA-RNA, a DNA-DNA, or RNA-DNA duplex is formed. These nucleic acids are often termed “antisense” because they are complementary to the sense or coding strand of the gene. Recently, formation of a triple helix has proven possible where the oligonucleotide is bound to a DNA duplex. It was found that oligonucleotides could recognise sequences in the major groove of the DNA double helix. A triple helix was formed thereby. This suggests that it is possible to synthesise a sequence-specific molecules which specifically bind double-stranded DNA via recognition of major groove hydrogen binding sites.

By binding to the target nucleic acid, the above oligonucleotides can inhibit the function of the target nucleic acid. This could, for example, be a result of blocking the transcription, processing, poly(A) addition, replication, translation, or promoting inhibitory mechanisms of the cells, such as promoting RNA degradations.

Antisense oligonucleotides are prepared in the laboratory and then introduced into cells, for example by microinjection or uptake from the cell culture medium into the cells, or they are expressed in cells after transfection with plasmids or retroviruses or other vectors carrying an antisense gene. Antisense oligonucleotides were first discovered to inhibit viral replication or expression in cell culture for Rous sarcoma virus, vesicular stomatitis virus, herpes simplex virus type 1, simian virus and influenza virus. Since then, inhibition of mRNA translation by antisense oligonucleotides has been studied extensively in cell-free systems including rabbit reticulocyte lysates and wheat germ extracts. Inhibition of viral function by antisense oligonucleotides has been demonstrated in vitro using oligonucleotides which were complementary to the AIDS HIV retrovirus RNA (Goodchild, J. 1988 “Inhibition of Human Immunodeficiency Virus Replication by Antisense Oligodeoxynucleotides”, Proc. Natl. Acad. Sci. (USA) 85(15), 5507-11). The Goodchild study showed that oligonucleotides that were most effective were complementary to the poly(A) signal; also effective were those targeted at the 5′ end of the RNA, particularly the cap and 5′ untranslated region, next to the primer binding site and at the primer binding site. The cap, 5′ untranslated region, and poly(A) signal lie within the sequence repeated at the ends of retrovirus RNA (R region) and the oligonucleotides complementary to these may bind twice to the RNA.

Oligonucleotides are subject to being degraded or inactivated by cellular endogenous nucleases. To counter this problem, it is possible to use modified oligonucleotides, eg having altered internucleotide linkages, in which the naturally occurring phosphodiester linkages have been replaced with another linkage. For example, Agrawal et al (1988) Proc. Natl. Acad. Sci. USA 85, 7079-7083 showed increased inhibition in tissue culture of HIV-1 using oligonucleotide phosphoramidates and phosphorothioates. Sarin et al (1988) Proc. Natl. Acad. Sci. USA 85, 7448-7451 demonstrated increased inhibition of HIV-1 using oligonucleotide methylphosphonates. Agrawal et at (1989) Proc. Natl. Acad. Sci. USA 86, 7790-7794 showed inhibition of HIV-1 replication in both early-infected and chronically infected cell cultures, using nucleotide sequence-specific oligonucleotide phosphorothioates. Leither et al (1990) Proc. Natl. Acad. Sci. USA 87, 3430-3434 report inhibition in tissue culture of influenza virus replication by oligonucleotide phosphorothioates.

Oligonucleotides having artificial linkages have been shown to be resistant to degradation in vivo. For example, Shaw et al (1991) in Nucleic Acids Res. 19, 747-750, report that otherwise unmodified oligonucleotides become more resistant to nucleases in vivo when they are blocked at the 3′ end by certain capping structures and that uncapped oligonucleotide phosphorothioates are not degraded in vivo.

A detailed description of the H-phosphonate approach to synthesising oligonucleoside phosphorothioates is provided in Agrawal and Tang (1990) Tetrahedron Letters 31, 7541-7544, the teachings of which are hereby incorporated herein by reference. Syntheses of oligonucleoside methylphosphonates, phosphorodithioates, phosphoramidates, phosphate esters, bridged phosphoramidates and bridge phosphorothioates are known in the art. See, for example, Agrawal and Goodchild (1987) Tetrahedron Letters 28, 3539; Nielsen et al (1988) Tetrahedron Letters 29, 2911; Jager et al (1988) Biochemistry 27, 7237; Uznanski et al (1987) Tetrahedron Letters 28, 3401; Bannwarth (1988) Helv. Chim. Acta. 71, 1517; Crosstick and Vyle (1989) Tetrahedron Letters 30, 4693; Agrawal et al (1990) Proc. Natl. Acad. Sci. USA 87, 1401-1405, the teachings of which are incorporated herein by reference. Other methods for synthesis or production also are possible. In a preferred embodiment the oligonucleotide is a deoxyribonucleic acid (DNA), although ribonucleic acid (RNA) sequences may also be synthesised and applied.

The oligonucleotides useful in the invention preferably are designed to resist degradation by endogenous nucleolytic enzymes. In vivo degradation of oligonucleotides produces oligonucleotide breakdown products of reduced length. Such breakdown products are more likely to engage in non-specific hybridization and are less likely to be effective, relative to their full-length counterparts. Thus, it is desirable to use oligonucleotides that are resistant to degradation in the body and which are able to reach the targeted cells. The present oligonucleotides can be rendered more resistant to degradation in vivo by substituting one or more internal artificial internucleotide linkages for the native phosphodiester linkages, for example, by replacing phosphate with sulphur in the linkage. Examples of linkages that may be used include phosphorothioates, methylphosphonates, sulphone, sulphate, ketyl, phosphorodithioates, various phosphoramidates, phosphate esters, bridged phosphorothioates and bridged phosphoramidates. Such examples are illustrative, rather than limiting, since other internucleotide linkages are known in the art. See, for example, Cohen, (1990) Trends in Biotechnology. The synthesis of oligonucleotides having one or more of these linkages substituted for the phosphodiester internucleotide linkages is well known in the art, including synthetic pathways for producing oligonucleotides having mixed internucleotide linkages. Oligonucleotides can be made resistant to extension by endogenous enzymes by capping or incorporating similar groups on the 5′ or 3′ terminal nucleotides. A reagent for capping is commercially available as Amino-Link II™ from Applied BioSystems Inc, Foster City, Calif. Methods for capping are described, for example, by Shaw et al (1991) Nucleic Acids Res. 19, 747-750 and Agrawal et al (1991) Proc. Natl. Acad. Sci. USA 88(17), 7595-7599, the teachings of which are hereby incorporated herein by reference.

A further method of making oligonucleotides resistant to nuclease attack is for them to be “self-stabilised” as described by Tang et al (1993) Nucl. Acids Res. 21, 2729-2735 incorporated herein by reference. Self-stabilised oligonucleotides have hairpin loop structures at their 3′ ends, and show increased resistance to degradation by snake venom phosphodiesterase, DNA polymerase I and fetal bovine serum. The self-stabilised region of the oligonucleotide does not interfere in hybridization with complementary nucleic acids, and pharmacokinetic and stability studies in mice have shown increased in vivo persistence of self-stabilised oligonucleotides with respect to their linear counterparts.

It is preferred that the antisense reagent is able to bind to nucleic acid encoding a receptor that mediates endotoxin (LPS) signalling, for example CD14 or toll-like receptors, particularly toll-like receptor 4 and 2.

The antisense compound may be administered systemically. The oligonucleotides also can be incorporated into an implantable device which when placed at the desired site, permits the oligonucleotides to be released into the surrounding locus. For example, implants made of biodegradable materials such as polyanhydrides, polyorthoesters, polylactic acid and polyglycolic acid and copolymers thereof, collagen, and protein polymers, or non-biodegradable materials such as ethylenevinyl acetate (EVAc), polyvinyl acetate, ethylene vinyl alcohol, and derivatives thereof can be used to locally deliver the oligonucleotides. The oligonucleotides can be incorporated into the material as it is polymerised or solidified, using melt or solvent evaporation techniques, or mechanically mixed with the material. In one embodiment, the oligonucleotides are mixed into or applied onto coatings for implantable devices such as dextran coated silica beads, stents, or catheters.

The dose of oligonucleotides is dependent on the size of the oligonucleotides and the purpose for which is it administered. In general, the range is calculated based on the surface area of tissue to be treated. The effective dose of oligonucleotide is somewhat dependent on the length and chemical composition of the oligonucleotide but is generally in the range of about 30 to 3000 μg per square centimetre of tissue surface area.

The oligonucleotides may be administered to the patient systemically for both therapeutic and prophylactic purposes. The oligonucleotides may be administered by any effective method, for example, parenterally (eg intravenously, subcutaneously, intramuscularly) or by oral, nasal or other means which permit the oligonucleotides to access and circulate in the patient's bloodstream. Oligonucleotides administered systemically preferably are given in addition to locally administered oligonucleotides, but also have utility in the absence of local administration. dosage in the range of from about 0.1 to about 10 grams per administration to an adult human generally will be effective for this purpose.

It will be appreciated that it may be desirable to target the antisense oligonucleotides to immune system cells, for example mononuclear phagocytes. This may be achieved by using antisense oligonucleotides which are in association with a molecule which selectively directs the antisense oligonucleotide to the immune system cells, for example mononuclear phagocytes. For example, the antisense oligonucleotide may be associated with an antibody or antibody like molecule which selectively binds an antigen present on appropriate immune system cells. Such antigens are well known to those skilled in the art. By “associated with” we mean that the antisense oligonucleotide and the immune cell-directing entity are so associated that the immune cell-directing entity is able to direct the antisense oligonucleotide to the immune system cells, for mononuclear phagocytes.

It will be appreciated that antisense agents also include larger molecules which bind to the receptor, for example CD14 mRNA or mRNA for toll-like receptors or genes and substantially prevent expression of the receptor, for example CD14 mRNA or mRNA for toll-like receptors or genes and substantially prevent expression of said receptor, for example CD14 protein. Thus, expression of an antisense molecule which is substantially complementary to the receptor, for example CD14 mRNA or mRNA for toll-like receptors is envisaged as part of the invention.

The said larger molecules may be expressed from any suitable genetic construct as is described below and delivered to the patient. Typically, the genetic construct which expresses the antisense molecule comprises at least a portion of the said receptor, for example CD14, toll-like receptors, mRNA or gene operatively linked to a promoter which can express the antisense molecule in the immune system cell. Promoters that may be active in immune system cells, for example mononuclear phagocytic cells will be known to those skilled in the art, and may include promoters for ubiquitously expressed, for example housekeeping genes.

Although the genetic construct can be DNA or RNA it is preferred if it is DNA.

Preferably, the genetic construct is adapted for delivery to a human cell.

Means and methods of introducing a genetic construct into a cell in an animal body are known in the art. For example, the constructs of the invention may be introduced into the tumour cells by any convenient method, for example methods involving retroviruses, so that the construct is inserted into the genome of the tumour cell. For example, in Kuriyama et al (1991) Cell Struc. and Func. 16, 503-510 purified retroviruses are administered. Retroviruses provide a potential means of selectively infecting cancer cells because they can only integrate into the genome of dividing cells; most normal cells surrounding cancers are in a quiescent, non-receptive stage of cell growth or, at least, are dividing much less rapidly than the tumour cells. Retroviral DNA constructs which encode said antisense agents may be made using methods well known in the art. To produce active retrovirus from such a construct it is usual to use an ecotropic psi2 packaging cell line grown in Dulbecco's modified Eagle's medium (DMEM) containing 10% foetal calf serum (FCS). Transfection of the cell line is conveniently by calcium phosphate co-precipitation, and stable transformants are selected by addition of G418 to a final concentration of 1 mg/ml (assuming the retroviral construct contains a neo^{R} gene). Independent colonies are isolated and expanded and the culture supernatant removed, filtered through a 0.45 μm pore-size filter and stored at −70°. For the introduction of the retrovirus into the tumour cells, it is convenient to inject directly retroviral supernatant to which 10 μg/ml Polybrene has been added. For tumours exceeding 10 mm in diameter it is appropriate to inject between 0.1 ml and 1 ml of retroviral supernatant; preferably 0.5 ml.

Alternatively, as described in Culver et al (1992) Science 256, 1550-1552, cells which produce retroviruses are injected into the tumour. The retrovirus-producing cells so introduced are engineered to actively produce retroviral vector particles so that continuous productions of the vector occurred within the tumour mass in situ. Thus, proliferating tumour cells can be successfully transduced in vivo if mixed with retroviral vector-producing cells.

Targeted retroviruses are also available for use in the invention; for example, sequences conferring specific binding affinities may be engineered into preexisting viral env genes (see Miller & Vile (1995) Faseb J. 9, 190-199 for a review of this and other targeted vectors for gene therapy).

Other methods involve simple delivery of the construct into the cell for expression therein either for a limited time or, following integration into the genome, for a longer time. An example of the latter approach includes (preferably tumour-cell-targeted) liposomes (Nassander et al (1992) Cancer Res. 52, 646-653).

Immunoliposomes (antibody-directed liposomes) are especially useful in targeting to cancer cell types which over-express a cell surface protein for which antibodies are available. For the preparation of immuno-liposomes MPB-PE (N-[4-(p-maleimidophenyl)butyryl]-phosphatidylethanolamine) is synthesised according to the method of Martin & Papahadjopoulos (1982) J. Biol. Chem. 257, 286-288. MPB-PE is incorporated into the liposomal bilayers to allow a covalent coupling of the antibody, or fragment thereof, to the liposomal surface. The liposome is conveniently loaded with the DNA or other genetic construct of the invention for delivery to the target cells, for example, by forming the said liposomes in a solution of the DNA or other genetic construct, followed by sequential extrusion through polycarbonate membrane filters with 0.6 μm and 0.2 μm pore size under nitrogen pressures up to 0.8 MPa. After extrusion, entrapped DNA construct is separated from free DNA construct by ultracentrifugation at 80 000×g for 45 min. Freshly prepared MPB-PE-liposomes in deoxygenated buffer are mixed with freshly prepared antibody (or fragment thereof) and the coupling reactions are carried out in a nitrogen atmosphere at 4 C under constant end over end rotation overnight. The immunoliposomes are separated from unconjugated antibodies by ultracentrifugation at 80 000×g for 45 min. Immunoliposomes may be injected intraperitoncally or directly into the tumour.

Other methods of delivery include adenoviruses carrying external DNA via an antibody-polylysine bridge (see Curiel Prog. Med. Virol. 40, 1-18) and transferrin-polycation conjugates as carriers (Wagner et al (1990) Proc. Natl. Acad. Sci. USA 87, 3410-3414). In the first of these methods a polycation-antibody complex is formed with the DNA construct or other genetic construct of the invention, wherein the antibody is specific for either wild-type adenovirus or a variant adenovirus in which a new epitope has been introduced which binds the antibody. The polycation moiety binds the DNA via electrostatic interactions with the phosphate backbone. The adenovirus, because it contains unaltered fibre and penton proteins, is internalised into the cell and carries into the cell with it the DNA construct of the invention. It is preferred if the polycation is polylysine.

The DNA may also be delivered by adenovirus wherein it is present within the adenovirus particle, for example, as described below.

In the second of these methods, a high-efficiency nucleic acid delivery system that uses receptor-mediated endocytosis to carry DNA macromolecules into cells is employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to polylysines of various sizes through a disulfide linkage. These modified transferrin molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilobase pairs). When complexes of transferrin-polycation and the DNA constructs or other genetic constructs of the invention are supplied to the tumour cells, a high level of expression from the construct in the cells is expected. High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. This approach appears to rely on the fact that adenoviruses are adapted to allow release of their DNA from an endosome without passage through the lysosome, and in the presence of, for example transferrin linked to the DNA construct or other genetic construct of the invention, the construct is taken up by the cell by the same route as the adenovirus particle.

This approach has the advantages that there is no need to use complex retroviral constructs; there is no permanent modification of the genome as occurs with retroviral infection; and the targeted expression system is coupled with a targeted delivery system, thus reducing toxicity to other cell types.

It will be appreciated that “naked DNA” and DNA complexed with cationic and neutral lipids may also be useful in introducing the DNA into cells of the patient to be treated. Non-viral approaches to gene therapy are described in Ledley (1995) Human Gene Therapy 6, 1129-1144.

Alternative targeted delivery systems are also known such as the modified adenovirus system described in WO 94/10323 wherein, typically, the DNA is carried within the adenovirus, or adenovirus-like, particle. Michael et al (1995) Gene Therapy 2, 660-668 describes modification of adenovirus to add a cell-selective moiety into a fibre protein. Mutant adenoviruses which replicate selectively in p53-deficient human tumour cells, such as those described in Bischoff et al (1996) Science 274, 373-376 are also useful for delivering the genetic construct of the invention to a cell. Thus, it will be appreciated that a further aspect of the invention provides a virus or virus-like particle comprising a genetic construct of the invention. Other suitable viruses or virus-like particles include HSV, AAV, vaccinia and parvovirus.

In a further embodiment the agent which is able to inhibit the response by a cell to endotoxin (LPS) is a ribozyme capable of cleaving targeted receptor, for example CD14, toll-like receptors, RNA or DNA. A gene expressing said ribozyme may be administered in substantially the same and using substantially the same vehicles as for the antisense molecules.

Ribozymes which may be encoded in the genomes of the viruses or virus-like particles herein disclosed are described in Cech and Herschlag “Site-specific cleavage of single stranded DNA” U.S. Pat. No. 5,180,818; Altman et al “Cleavage of targeted RNA by RNAse P” U.S. Pat. No. 5,168,053, Cantin et al “Ribozyme cleavage of HIV-1 RNA” U.S. Pat. No. 5,149,796; Cech et al “RNA ribozyme restriction endoribonucleases and methods”, U.S. Pat. No. 5,116,742; Been et al “RNA ribozyme polymerases, dephosphorylases, restriction endonucleases and methods”, U.S. Pat. No. 5,093,246; and Been et al “RNA ribozyme polymerases, dephosphorylases, restriction endoribonucleases and methods; cleaves single-stranded RNA at specific site by transesterification”, U.S. Pat. No. 4,987,071, all incorporated herein by reference.

It will be appreciated that it may be desirable that the antisense molecule or ribozyme is expressed from a immune system cell-specific promoter element.

The genetic constructs described above can be prepared using methods well known in the art. The compound may inhibit signalling via the receptor, for example the CD14 or toll-like receptors. The compound may be an antibody that binds to CD14 or toll-like receptors and reduces its signalling activity. A suitable antibody may be described in U.S. Pat. No. 5,730,980.

It is preferred that the compound is able to substantially reduce the amount of immune mediators produced in response to the presence of endotoxin (LPS).

It will be appreciated that the agent administered to the patient may be a single chemical species, or it may be a mixture of two or more chemical species.

The compound may be administered to the patient in any suitable form or in any suitable way. The compound or a formulation thereof may be administered by any conventional method including oral and by injection (in particular, intravascular injection). The treatment may consist of a single dose or a plurality of doses over a period of time.

Activated charcoal may be administered as a slurry in water, as well known to those skilled in the art, but additives may be desirable in order to improve the flavour and texture. Suitable additives and formulations are described in Martindale: The Extra Pharmacopoeia, 31^{st} edition. Activated charcoal may also be presented as granules, tablets or biscuits.

Chronic use is suggested in any patient who is at increased risk of myocardial infarction (i.e. any patient with coronary artery disease—all at risk for acute heart failure) or in any patient with chronic heart failure (at risk for decompensation and cachexia development).

While it is possible for the compound to be administered alone, it is preferable to present it as a pharmaceutical formulation, together with one or more acceptable carriers. The carrier(s) must be “acceptable” in the sense of being compatible with the compound and not deleterious to the recipients thereof.

The formulations may conveniently be presented in unit dosage form and may be prepared by any of the methods well known in the art of pharmacy. Such methods include the step of bringing into association the compound (active ingredient) with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations in accordance with the present invention suitable for oral administration may be presented as discrete units such as capsules, sachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. An enteric coated formulation may be useful in delivering the agent to the lower gastrointestinal tract, for example the bowel. The active ingredient may also be present as a bolus electuary or paste.

A tablet may be made by compression or moulding, optionally with one or more accessory ingredients. Compressed tablets may be prepared by compressing in a suitable machine the active ingredient in a free-flowing form such as a powdered or granules, optionally mixed with a binder (eg povidone, gelatin, hydroxypropylmethyl cellulose), lubricant, inert diluent, preservative, disintegrant (eg sodium starch glycollate, cross-linked povidone, cross-linked sodium carboxymethyld cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for parenteral administration include aqueous and non-aqueous sterile injection solutions which may contain anti-oxidants, buffers, bacteriostats and solutes which render the formation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

It will be appreciated that intravascular administration may be particularly desirable in the treatment of acute heart failure, for example where there is a desire for the avoidance of resorption loss of the bile acid and for a quicker onset of action.

A further aspect of the invention provides use of:
a compound that is able to bind to an endotoxin (lipopolysaccharide; LPS) molecule,
a compound that is able to bind to an endotoxin (lipopolysaccharide; LPS) molecule in the gut,
a antibacterial agent (that is preferably active in the gut),
a compound that is able to inhibit the response by a cell to endotoxin (LPS)
an agent that is able to reduce the permeability of the gut wall to bacteria and/or endotoxin (LPS)
   in the manufacture of a medicament for treating, preventing or ameliorating endotoxin-mediated immune activation in acute or chronic heart failure in a patient. Preferences for the said compound are as set out above.

A further aspect of the invention provides a pharmaceutical formulation comprising a compound as defined above and a diuretic. A still further aspect of the invention provides a kit of parts useful in treating, preventing or ameliorating acute or chronic heart failure comprising a compound as defined above and a diuretic. A diuretic may be administered to the patient to whom the method or use of any of the preceding aspects of the invention relates.

Suitable diuretics are known to those skilled in the art and are described, for example in Martindale The Extra Pharmacopoeia, 31^{st} Edition.

A further aspect of the invention provides any novel method of treating, preventing or ameliorating acute or chronic heart failure as herein disclosed.

The present invention also relates to therapy and the use of agents in the therapy of cachexia and wasting syndromes due to diseases other than congestive heart failure. Cachexia occurs in a number of other chronic diseases, like liver cirrhosis, chronic obstructive pulmonary disease, chronic renal failure, diabetes, rheumatoid arthritis. Cachexia and weight loss are linked to inflammatory processes and they are linked to increased mortality and/or morbidity. Cytokine activation is a potential causal mechanism for the development of cachexia also in these other diseases.

No one has previously proposed that one or all of the following agents may be useful in the management of patients with cachexia due to liver cirrhosis, chronic obstructive pulmonary disease, chronic renal failure, diabetes, rheumatoid arthritis:
a bile acid,
BPI,
LPS binding protein or a functional equivalent thereof
an antibody capable of binding to endotoxin,
the combination of lipoproteins and LPS binding protein
activated charcoal, Fuller's earth, attapulgite, kaolin or bentonite or a clay,
an antibody able to bind the CD14 receptor,
a soluble CD14 receptor,
a drug blocking effectively signaling through toll-like receptors, particularly toll-like receptor 4 and 2
colostrum of human, bovine, or other mamallian origin

The following classes of patients in particular may benefit from treatment

1. Patients with liver cirrhosis, chronic obstructive pulmonary disease, chronic renal failure, diabetes, rheumatoid arthritis.
2. Patients with cachexia due to liver cirrhosis, chronic obstructive pulmonary disease, chronic renal failure, diabetes, rheumatoid arthritis.

It is preferred that the patient has cachexia, as characterised by loss of muscle, fat, and or bone tissue.

It is preferred that the patient has experienced weight loss >7.5%.

It is preferred that the compound is able to substantially reduce the biological activity of endotoxin (lipopolysaccharide) such that the endotoxin mediated production of inflammatory cytokines in the circulating blood is reduced.

By “bile acid” we include all naturally occurring bile acids whether from man or from another animal. Also is included bile acids which are synthetic or semi-synthetic derivatives of naturally occurring bile acids. Of course, all bile acids including those that are “naturally occurring” may be synthesised chemically.

Bile acids are available from Falk Pharma GmbH and are described, for example, in WP96/17859, DE29717252 and WO98/05339.

Bile acids for use in the method of the invention include, but are not limited to, chemodeoxycholic acid (3α,7α-dihydroxy-5-cholan-24-oic acid), arsodeoxycholic acid (3α, 7-dihydroxy-5-cholan-24-oic acid), dehydrocholic acid (3,7,12-trioxo-5-cholan-24-oic acid), cholic acid and deoxycholic acid.

Preferably, the bile acid is a bile acid which is able to form micelles. Preferably, the bile acid is able to form a micelle around an endotoxin (lipopolysacharide molecule). It is particularly preferred that the bile acid is able to bind to endotoxin (lipopolysaccharide) molecules and substantially reduce the available endotoxin in the patient. In particular, it is preferred if the bile acid is able to substantially reduce the biological activity of endotoxin (lipopolysaccharide) such that the endotoxin has a substantially reduced effect on the liver or does not reach the liver in a substantially active form.

It is preferred if the bile acid is any one of ursodeoxycholic acid, chemodeoxycholic acid, dehydrocholic acid, cholic acid and deoxycholic acid.

It is preferred if the bile acid is ursodeoxycholic acid.

Originally, UDCA was registered for the medical treatment of gallstones (Leuschner et al. Our ten year experience in gallstone dissolution. Comparison with the national Canadian gallstone (NCGS, USA) and the Toky co-operative gallstone study (TCGS, Japan). Gastroenterology 1982, 82:1113). Ursodeoxycholic acid has for many years been proposed to be useful also in patients with cholestatic disease, and particularly in patients with primary biliary cirrhosis, a chronic cholestatic liver disease (Lindor et al. Effects of ursodeoxycholic acid on survival in patients with primary biliary cirrhosis. Gastroenterology 1996, 110:1515-1518). In analogy, UDCA is used in other cholestatic disorders like primary sclerosing cholangitis (Beuers et al: Therapie der autoimmunen Hepatitis, primär biliären Zirrhose und primär sklerosierenden Cholangitis. Konsensus der Deutschen Geselilschaft für Verdauungs-und Stoffwechselkrankheiten. Z. Gastroenterologie 1997; 35:1041-1049) or benign cholestasis of pregnancy (Palma et al. Ursodeoxycholic acid in the treatment of cholestasis of pregnancy: a randomized, double-blind study controlled with placebo. J Hepatol 1997, 27:1022-1028). Regarding its mode of action, most authorities regard increased bile flow and a reduced hepatocellular insult as a result of improved bile flow and altered bile salt patterns as the main modes of UDCA action in chronic cholestatic liver diseases.

However, a very recent meta-analysis concluded that “Published randomised controlled trials of UDCA do not show evidence of therapeutic benefit in primary biliary cirrhosis and its use as standard therapy needs to be re-examined.” (Goulis et al. Randomised controlled trials of ursodeoxycholic-acid therapy for primary biliary cirrhosis: a meta-analysis. Lancet 1999 Sep. 25; 354:1053-1060.)

As for other liver diseases another recent review article concluded “Ursodeoxycholic acid is of unproven efficacy in non-cholestatic disorders such as acute rejection after liver transplantation, non-alcoholic steatohepatitis, alcoholic liver disease and chronic viral hepatitis.” Trauner M and Graziadei I W. Review article: mechanisms of action and therapeutic applications of ursodeoxycholic acid in chronic liver diseases. Aliment Pharmacol Ther. 1999 August; 13(8): 979-996.

Therefore, treatment with ursodeoxycholic acid (UDCA) can not be considered a treatment with proven efficacy in patients with liver disease.

It has never been suggested that ursodeoxycholic acid (UDCA) should be specifically given to patients with cachexia due to liver cirrhosis.

It has never been suggested that ursodeoxycholic acid (UDCA) should be specifically given to patients with alcoholic liver cirrhosis. In fact, such patients were specifically excluded from studies.

Alterations in nutritional state leading to abnormal body composition are detectable already in early stages of liver cirrhosis and are clinically overt in the great majority of patients with advanced disease. Despite the well accepted prognostic role of cachexia or protein-energy-malnutrition in cirrhosis its pathogenesis is not fully understood. Although alcohol abuse and inadequate nutrient composition may play some role in patients with alcoholic liver disease this clearly is not operative in patients with liver disease of other etiology in whom malnutrition is as great a problem as in those with alcoholic liver disease (Plauth et al: ESPEN guidelines for nutrition in liver disease and transplantation. Clin Nutr 1997, 16:43-55). Nutrient intake is reduced in many patients with advanced liver cirrhosis and does not match requirements. It is unknown, however, whether food intake is reduced as a consequence of mechanical factors such as ascites or due to altered appetite regulation or other processes.

It is long known that endotoxaemia occurs in a number of patients with liver cirrhosis. It is not known, whether endotoxin (LPS) levels are particularly raised in patients with cachexia due to liver cirrhosis.

Depending of the severity of the liver cirrhosis process, cachexia occurs in 30 to 60% of patients with liver cirrhosis, and the survival of patients with cachexia in liver cirrhosis is impaired. (Plauth et al: ESPEN guidelines for nutrition in liver disease and transplantation. Clin Nutr 1997, 16:43-55). There is no known specific therapy for these patients, and randomised placebo controlled clinical trials to reverse the cachexia in liver cirrhosis patients, and particularly in those with alcohol induced liver cirrhosis have not been performed. Additionally, patients with a body cell mass (BCM)<35% of body weight have reduced survival also after liver transplantation, and the 5-year survival rate is 54% compared to 88% in patients with BCM >35% (p<0.01) (Selberg et al. Identification of high- and low-risk patients before liver transplantation: a prospective cohort study of nutritional and metabolic parameters in 150 patients. Hepatology 1997; 25:652-657).

It has also been suggested that bile acids can protect the liver against endotoxin action in obstructive jaundice when patients undergo surgery (Greve et al. Bile acids inhibit endotoxin-induced release of tumor necrosis factor by monocytes: an in vitro study. Hepatology 1989 October; 10(4):454-458). With regards to monocyte generated cytokine production in response to LPS, in this study deoxycholic acid was the most effective, chenodeoxycholic acid was less effective and ursodeoxycholic acid was ineffective in the concentrations used. Bile acids did not inactivate endotoxin as measured in a chromogcnic Limulus amebocyte lysate assay. In these studies patients with non-cholestatic or alcoholic aetiology were not considered, and there was no data or discussion of cachexia and weight loss.

In experiments, rats with obstructive jaundice, LPS was administered via the portal vein. In UDCA-treated rats, the endotoxin concentration was significantly lower, however, that UDCA had no effect on the TNF-alpha levels (Hori Y & Ohyanagi H. Protective effect of the intravenous administration of ursodeoxycholic acid against endotoxaemia in rats with obstructive jaundice. Surg-Today 1997; 27:140-144). In a case control study UDCA showed also no clinical benefit in patients with chronic hepatitis C, and serum TNF and IL-6 levels could not be shown to be affected by UDCA treatment (Lu et al. Efficacy of ursodeoxycholic acid in the treatment of patients with chronic hepatitis C. J Gastroenterol Hepatol 1995; 10:432-437.

In summary, the immunological effects of ursodeoxycholic acid (UDCA) on plasma LPS and cytokine levels are poor in these studies, and the cellular effects of ursodeoxycholic acid (UDCA) are conflicting.

It is important to note that it has never been proposed that ursodeoxycholic acid (UDCA) should be given in patients with weight loss, i.e. cachexia, in patients with liver disease. It has never been proposed that ursodeoxycholic acid (UDCA) could prevent or reverse weight loss, i.e. cachexia, in patients with liver disease. Additionally, it has never been proposed that ursodeoxycholic acid (UDCA) could prevent or reverse weight loss, i.e. cachexia, in patients with chronic obstructive pulmonary disease, chronic renal failure, diabetes, rheumatoid arthritis.

The invention as detailed so far will now be described by reference to the following Examples and Figures:

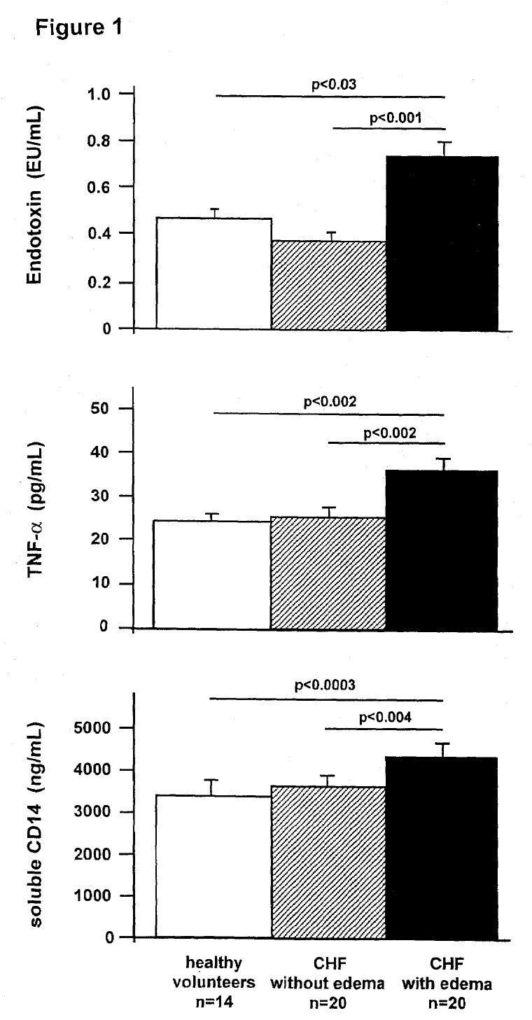

FIG. 1: Plasma levels of endotoxin, TNFα and soluble CD14 in patients with chronic heart failure (CHF) with and without peripheral edema compared to healthy volunteers (mean±standard error of the mean).

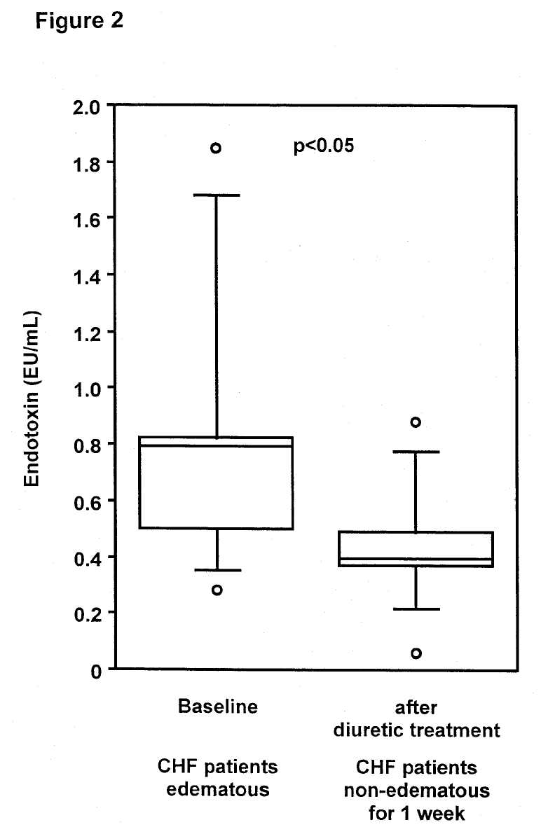

FIG. 2: Effect of intensified diuretic treatment on plasma endotoxin levels in 10 CHF patients with peripheral edema (box plot displaying the 10^{th}, 25^{th}, 50^{th} and 90^{th} percentiles).

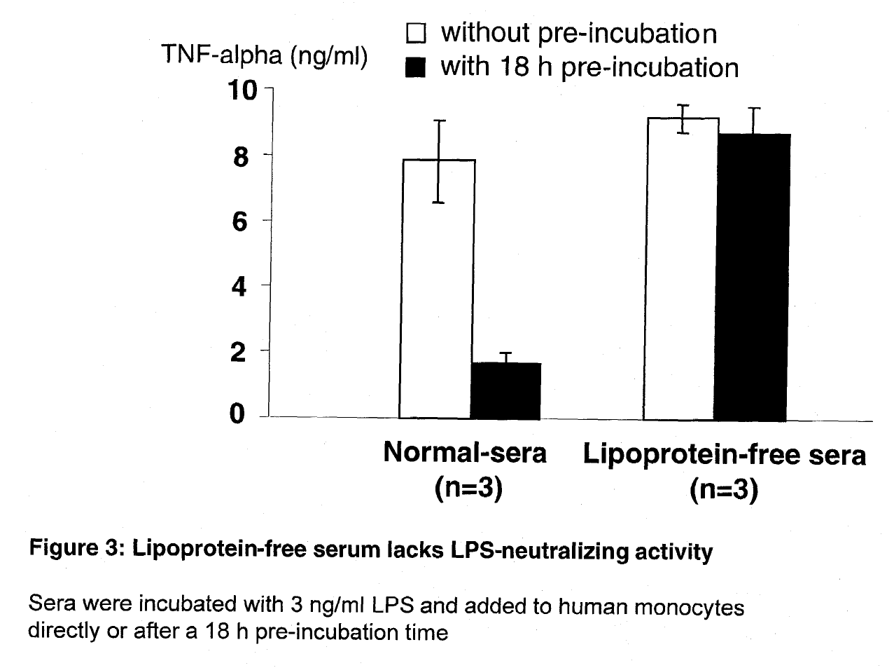

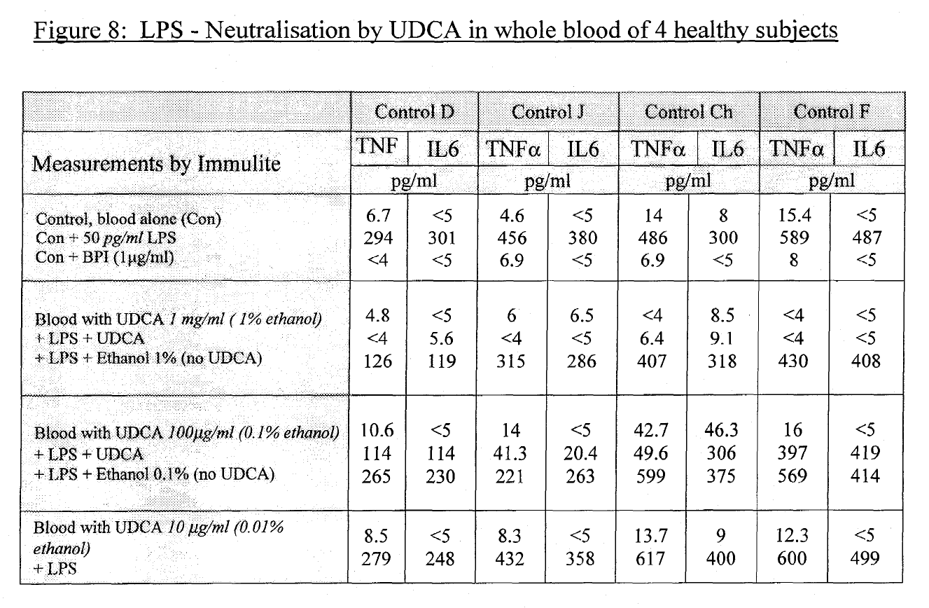

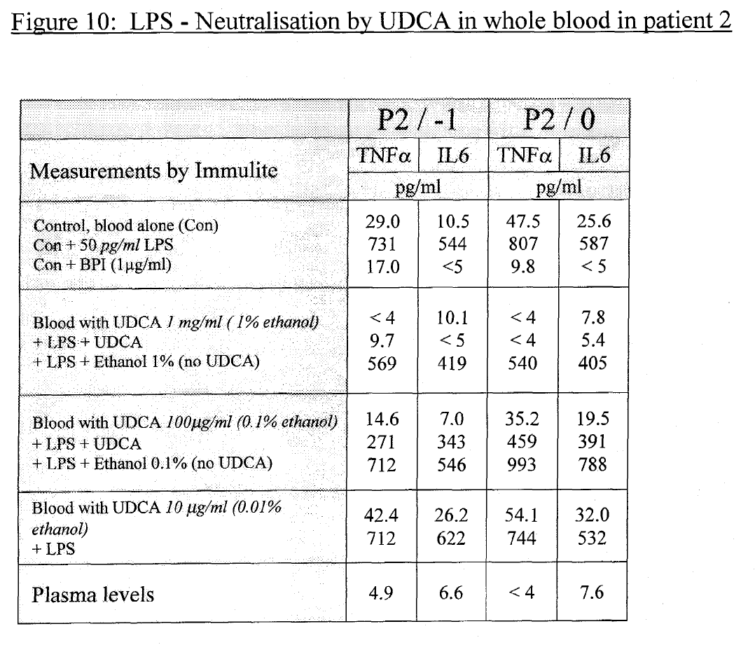

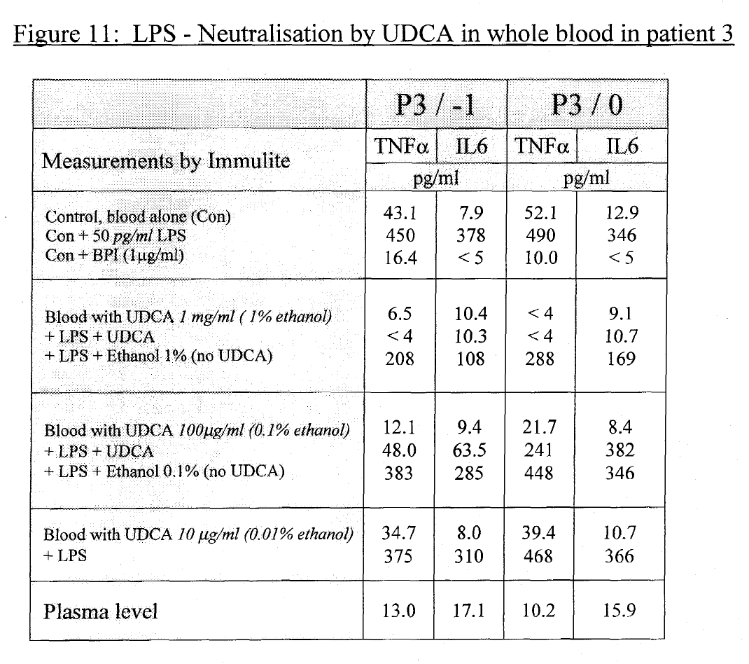

FIG. 3 to 12

### EXAMPLE 1

### Endotoxin and Immune Activation in Chronic Heart Failure

### Summary

Background: This study was designed to test the hypothesis that endotoxemia occurs during the congestive phase of CHF. Immune activation in chronic heart failure (CHF) patients may be secondary to endotoxin action.

Methods: We studied 20 CHF patients with recent onset of moderate to severe peripheral oedema secondary to cardiac congestion (age 64±2 y, NYHA class 3.3±0.1, mean±SEM) and compared them to 20 stable CHF patients (63±4 y, NYHA 2.6±0.2), and 14 healthy control subjects (55±4 y, ANOVA p=0.28). Blood samples for endotoxin measurements (LAL test, normal level <0.50 IU/mL) were collected in endotoxin free tubes. Biochemical markers of endotoxemia and inflammation, several cytokines and cell membrane proteins associated with immune activation were also measured. Ten patients were restudied within 1 week of complete resolution of oedema (5 patients survived >6 months and were restudied again).
Findings: Endotoxin levels were increased in oedematous CHF patients (0.74±0.10 IU/mL) as compared to stable CHF (0.3710.05 IU/mL, p=0.0009) and controls (0.4610.05 IU/mL, p=0.02); LPS binding protein (LBP) did not differ between groups. Compared to controls and stable CHF, oedematous CHF had highest levels of c-reactive protein (CRP, ANOVA p<0.003), tumor necrosis factor (TNF)-α (p<0.001), soluble (s) TNF receptor (—R)1 (p<0.001), sTNF-R2 (p<0.01), interleukin-6 (p<0.003), and sCD14 (p<0.001). Endotoxin levels correlated with sCD14 (r=0.30, p<0.03). CRP levels correlated with procalcitonin (r=0.74, p<0.0001), TNF-α (r=0.50, p=0.001), TNF-R1 (r=0.67, p<0.0001), and TNF-R2 (r=0.61, p<0.0001). FACS analyses revealed similar CD4/8 ratios in all groups, despite significantly reduced CD4 (p<0.02) and elevated CD8/25 (p<0.05) in CHF-oedema. Diuretic treatment with resolution of oedema resulted in normalisation of endotoxin levels after 23±8 days (n=10: 0.84±0.16 to 0.45±0.07 IU/mL, p<0.05), but cytokines remained elevated and LBP unchanged. After freedom of oedema>3 months endotoxin levels remained stable and normal (p=0.45, n=5), and TNF-α had decreased (39.6±5.5 to 31.0±2.5 pg/mL, p=0.079).

Interpretation: Elevated levels of endotoxin and cytokines without a concomitant increase in LBP are found in CHF patients during an acute oedematous exacerbation. Elevated endotoxin levels are normalised by intensified diuretic treatment, whereas normalisation of TNF-α levels is delayed. These data provide evidence for a role of endotoxin as a potential cause of immune activation in patients with congestive-heart failure.

The results show that LPS is raised in oedematous CHF, but normal in non-oedematous heart failure patients. The increased LPS levels are linked to raised cytokine levels. Diuretic treatment reduces LPS levels. This suggests that oedema may causally be linked with elevated LPS levels. After treating the oedema, cytokine levels (TNF etc.) but also levels of soluble CD14 (a marker of cell-LPS interaction) do not fall immediately. The cytokine levels fall only after a longer period of clinical stability. This suggests that LPS sensitivity may be abnormal in subjects after a phase of clinical instability, i.e. despite a “normal” level of LPS the interaction with immunological cells is still intensive (sCD14 is high) and cytokine production is still increased. LPS binding protein was not increased in any patient group.

Patients with chronic heart failure (CHF) exhibit immune activation which may be related to generalised body wasting (i.e. cardiac cachexia) [1,2]. Based on the finding of increased expression of tumor necrosis factor-α (TNF-α) in cardiac tissue of CHF patients undergoing heart transplantation the failing heart itself has been suggested as the cause of immune activation [3]. To date no link between a pathogenic process and cytokine activation in heart failure has been documented, either in patients with heart failure or animal models. The precise stimulus for the increased cytokine production seen in CHF patients remains unknown.

We have previously suggested that bacterial endotoxin, lipopolysaccharide (LPS), contributes to immune activation in CHF [4]. Acute venous congestion could cause immune activation via several mechanisms. Regional hypoxia could facilitate the generation of oxygen free radicals and altered gut permeability may lead to bacterial or LPS translocation. Alternatively, lung infection may be present. These events may increase LPS plasma levels and trigger increased cytokine production. LPS is bound by a serum protein termed LPS binding protein (LBP) [5], and it recently has been shown that the ratio of LPS to LBP is crucial for the immunostimulatory effects of LPS [6]. LBP levels in vivo can vary substantially due to transcriptional activation [7]. We have recently shown that high concentrations of LBP, as seen during the acute phase response, can completely block LPS effects in vitro and in a murine sepsis model [8]. Furthermore, in our previous study [4] to patients with high soluble (s) CD14 levels (indicative of endotoxin-cell interaction and shedding of CD14 from the cell membrane [9]) showed markedly increased levels of TNF-α, sTNF receptor (R)-1 and -2, and intercellular adhesion molecule-1 (ICAM-1). A recent report documented that sCD14 alone can stimulate immune cells to produce cytokines [10]. In the present study, we measured endotoxin, LBP and sCD14 and related levels to markers of cellular and humoral immune activation in CHF patients and healthy volunteers. Among CHF patients bowel wall oedema that could cause altered gut permeability and bacterial (ie endotoxin) translocation is most likely to occur in patients with moderate to severe peripheral oedema. Thus, we compared patients with recent onset oedematous decompensation to stable non-oedematous CHF patients. In a subgroup of oedematous patients we assessed the effect of diuretic therapy, anticipating that such treatment would lead to a reduction of endotoxin.

### Methods

Fourteen healthy volunteers (age: 5±54 y) and 40 CHF patients (age: 63±3 y, p=0.30) were studied prospectively. The aetiology of CHF was ischaemic in 27 patients and idiopathic dilated cardiomyopathy in 13 patients. The diagnosis of CHF was based on symptomatic exercise intolerance, cardiomegaly, and documented left ventricular dysfunction (all patients had a left ventricular ejection fraction of less than 40%). No subject had clinical signs of infection, rheumatoid arthritis, or cancer. Cardiac decompensation has been associated with the presence of bowel wall oedema secondary to venous congestion. We were not able to measure directly the degree of bowel wall oedema. The relationship between central haemodynamics and the pathophysiological alterations in CHF is weak [11,12]. In animal models there is a poor relationship between intracardiac pressures and intestinal perfusion [13]. Thus, we divided patients according to the presence or absence of a reliable marker of acute venous congestion due to cardiac failure, namely peripheral oedema.

Twenty CHF patients were clinically stable without evidence of peripheral oedema, and 20 patients presented with moderate to severe oedema to the outpatient clinic of the Royal Brompton Hospital in London, UK. The CHF patients were treated with diuretics (n=38), an angiotensin converting enzyme inhibitor (n=36), digoxin (n=14), aspirin (n=17), amiodarone (n=16) and nitrates (n=15) in varying combination. The clinical details of patients and controls are given in Table 1. Ten oedematous patients who lived close to our hospital (NYHA class IV: 5, class III: 5) were followed-up after treatment with increased doses of diuretics (increase of frusemide up to 120 mg/day, addition of bendrofluazide (2.5 or 5 mg od), and/or metolazone (5 or 10 mg od)). Of these patients three had to be admitted for 3 to 8 days for intravenous diuretic treatment. After 23-8 days these patients were restudied within 1 week after complete resolution of oedema (NYHA class after treatment: III-6, II-4; weight loss: 3.6±0.3 kg [range 2.5 to 5.0 kg]). Five patients regained clinical stability (NYHA class: III-1, II-4) and were restudied again 14 to 32 weeks (mean 21±3 weeks) after the initial investigation when they had been free of peripheral oedema for more then 3 months. The remaining 5 patients did not reach a longer-term stable clinical state again and died 2 to 8 months after the initial investigation without having been restudied. The research protocol was approved by the ethics committee of the Royal Brompton Hospital, and all patients and controls gave written informed consent.

Blood samples. Blood samples were collected on presentation in the outpatient clinic after supine rest for at least 15 min. An antecubital polyethylene catheter was inserted and 8 mL of venous blood were drawn into endotoxin free tubes (Endo Tube ET®, Chromogenix AB, Sweden), and 30 mL of standard venous samples were taken for biochemical and cytokine measurements. After immediate centrifugation endotubes and plasma aliquots were stored at −80° C. until analysis. In addition, 5 mL EDTA blood was taken to perform fluorescence activated cell sorting (FACS) analysis.

Assessment of endotoxin. Levels of endotoxin were measured by using a commercially available kit (Limulus Amebocyte Lysate QCL-1000 test kit, BioWhittaker Inc., Walkersville, USA). The normal level of endotoxin in this assay in healthy subjects is <0.50 IU/mL. Endotoxin in the patient sample activates a protein in the Limulus amebocyte lysate, so that it possesses enzymatic activity. The activated enzyme catalyses the release of p-nitroaniline from a short synthetic peptide; p-nitroaniline can be detected by acidification with acetic acid, and measuring absorbance at 410 nm (sensitivity 0.03 IU/mL). The coefficient of variance for the LPS reproducibility with the LAL test kit is <10%.

Cytokine and other analyses. LBP-levels were determined by an ELISA assay as described previously [14]. Total tumor necrosis factor (TNF)-α was measured with an ELISA test kit from Medgenix (Fleurus, Belgium; sensitivity 3.0 pg/mL; test not influenced by soluble TNF receptors). Soluble TNF receptors 1 (sTNF-R1; sensitivity 25 pg/mL), sTNF-R2 (sensitivity 2 pg/mL), and interleukin-6 (IL-6; sensitivity 0.0094 pg/mL, all kits: R&D Systems, Minneapolis, Minn., USA), and sCD14 (IBL, Hamburg, Germany) were assessed by ELISA. Plasma procalcitonin (PCT) levels were measured by an immunoluminometric assay using two monoclonal antibodies (BRAHMS, Berlin, Germany) [15,16]. The normal level of PCT in this assay in healthy subjects is <0.6 ng/ml.

FACS analysis. Whole blood samples were supplied for analysis in K-EDTA tubes (Vacutaner Systems, Falcon BD Oxford UK) and stained with fluorescently labeled monoclonal antibodies (Coulter Electronics, Luton UK) to determine peripheral lymphocyte phenotype and the proportion of CD25 receptor (CD25R) positive T cells. Briefly, a staining excess of antibody, determined by titration (data not shown), was aliquoted into 12×75 mm polypropylene tubes (Elkay, Hampshire UK). Two tubes were analysed for each patient sample point. The first contained control monoclonal mouse anti-human antibodies isotipically matched to the test antibodies in the second tube. The antibody-fluorochrome conjugates used were CD3-PC5, CD4-FITC, CD8-ECD, CD25R-RD1. The Immunoprep formic acid lysed whole blood protocol was used in the multi-Q-prep (Coulter Electronics, Luton, UK). Lymphocyte gating was set on forward versus side scatter dot plot and compensation established by combining single colour stained leukocyte populations. Four colour flow cytometric analysis was performed on the Coulter XL-MCL employing System II software.

Statistical analyses. Normality of distribution was assessed using the Kolmogorow Smirnov test. Unpaired Student's t-test, paired t-test, ANOVA with Fisher's post hoc test, and Mann-Whitney U test were used where appropriate. Data are presented as mean±standard error of the mean. We also performed univariate correlation analyses to establish the relationship between variables. A probability value of p<0.05 was considered significant.

### Results

Baseline analyses. In Table 1 and 2 baseline clinical characteristic and humoral measurements are detailed. Between controls and stable-CHF patients only uric acid and aspartate aminotransferase levels were significantly different. Oedematous CHF patients had more severe disease and showed a variety of biochemical abnormalities.

Endotoxin levels were highest in CHF patients with peripheral oedema (0.74±0.10 IU/mL) compared to CHF patients without oedema (0.37±0.05 IU/mL, p=0.0009), and controls (0.46±0.05 IU/mL, p=0.02) (FIG. 1). Plasma levels of LBP were not statistically different between groups (stable CHF:10.4±1.2 μg/mL, oedematous CHF:12.1±1.3 μg/mL, controls: 9.6±1.3 μg/mL), but there was an elevated LPS/log LBP ratio in the CHF patients with oedema (oedematous CHF:0.75±0.11, stable CHF:0.44±0.07, controls: 0.54±0.05, ANOVA p=0.03, oedematous CHF vs stable CHF:p<0.01). In oedematous CHF patients levels were highest for CRP (+107% vs stable CHF, p<0.03; +252% vs controls, p<0.001), TNF-α (+42% vs stable CHF, p<0.001; +49% vs controls, p<0.001, FIG. 1), sTNF-R1 (+78% vs stable CHF, p<0.006; +171% vs controls, p<0.0005), sTNFR-R2 (+50% vs stable CHF, p<0.03; +115% vs controls, p<0.001), IL-6 (+241% vs stable CHF, p<0.005; +635% vs controls, p<0.002) and sCD14 (+16% vs stable CHF, p<0.003; +23% vs controls, p<0.0003, FIG. 1). A trend toward increased PCT levels in oedematous CHF patients was noted (ANOVA: p=0.073).

Analysing the data of all subjects, there were significant correlations of sCD14 with endotoxin (r=0.30, p=0.028), as well as with TNF-α (r=0.36, p=0.008), sTNF-R1 (r=0.46, p=0.0005), and sTNF-R2 (r=0.38, p<0.009). CRP correlated with PCT (r=0.74, p<0.0001), TNF-α (r=0.49, p=0.001), sTNF-R1 (r=0.67, p<0.0001), and sTNF-R2 (r=0.61, p<0.0001), but not with endotoxin (r=0.09, p=0.57). Furthermore, PCT correlated with sTNF-R1 (r=0.50, p=0.0001) and sTNF-R2 (r=0.53, p<0.0001), but not with TNF-α (r=0.25, p=0.07) and endotoxin (r=0.03, p=0.83). There were neither simple correlations of creatinine or urea plasma levels and LPS at baseline, nor of changes of markers of kidney function over time vs the changes of LPS or cytokine concentrations over time (data not shown). Thus a bias due to latent abnormalities of kidney function seen in some oedematous patients is unlikely.

FACS analyses. There was significantly less CD4 in oedematous CHF patients (35±6%) as compared to stable-CHF (51±4%, p<0.007) and healthy volunteers (47±2%, p<0.03), whereas CD4/25 (CHF-oedema 10.6±3.3%, stable-CHF 5.5±0.7%, Con 6.7±1.1%, p>0.2), CD8 (CHF-oedema 28±8%, stable-CHF 23±5%, Con 22±2%, p>0.2), and the CD4/8 ratio (CHF-oedema 2.6±0.9%, stable-CHF 3.3±0.8%, Con 2.5±0.3%, p>0.2) were not different between groups. CD8/25 was significantly higher in patients with CHF-oedema (11.6±4.0%) than in healthy volunteers (4.7±0.6%, p<0.02), but not stable-CHF (8.7±1.6, p>0.2).

Influence of diuretic treatment. Intensive diuretic treatment of CHF patients (n=10) resulted in weight reduction of 3.6±0.3 kg (range 2.5 to 5.0 kg), and improvement of the functional NYHA class of 9 of the 10 patients. In 8 of 10 patients a reduction of the endotoxin plasma concentration by 17 to 90% was observed (mean for all patients: −46%); the LPS levels fell from 0.84±0.16 to 0.45±0.07 IU/mL (n=10, p<0.05; FIG. 2). In 2 patients with normal levels at baseline, endotoxin levels were found at the upper end of the normal range after diuretic treatment, i.e. below 0.50 IU/mL (+9% and +36% compared to baseline). Diuretic treatment did not affect plasma levels of TNF-α (baseline: 39.9±4.2 pg/mL, after: 40.2±4.1 pg/mL), sTNF-R1 (baseline: 2336±415 pg/mL, after: 2765±440 pg/mL), sTNF-R2 (baseline: 3751±378 pg/mL, after: 4029±437 pg/mL), IL-6 (baseline: 19.4±7.3 pg/mL, after: 18.3±7.6 pg/mL), sCD14 (baseline: 4474±70 ng/mL, after: 4430±241 ng/mL), or LBP (baseline: 10.3±1.2 μg/mL, after: 12.7±2.4 μg/mL) compared to baseline (n=10, all p>0.20). During further follow-up, 5 patients could be restudied when they had been free of oedema >3 months. Endotoxin remained stable at visit 3 (after 21±3 weeks: 0.49±0.03 IU/mL) compared to the second visit of these 5 patients (after 19±7 days: 0.39±0.10 IU/mL, p=0.45), but TNF-α decreased (visit 2: 39.6±5.5 vs visit 3: 31.0±2.5 pg/mL, p=0.079).

We have shown that endotoxin levels as well as pro-inflammatory cytokines are elevated in patients with heart failure who have peripheral oedema. Elevated endotoxin levels were normalised by prolonged diuretic treatment. The endotoxemia in these patients was not associated with a strong acute phase response that would have induced an increased hepatic LBP synthesis and subsequent blocking of LPS-effects. These results support the suggestion that bacterial endotoxin may be an important stimulus of immune activation in patients with chronic heart failure.

The complex of endotoxin and endotoxin binding protein activates cells via the CD14 protein on the surface of mononuclear phagocytes stimulating the production of TNF-α and other cytokines [17,18]. Previous studies suggested that increased sCD14 levels might be related to endotoxemia [9], but this is the first study to document directly the significant relationship between endotoxin and sCD14. Shedded and therefore soluble CD14 receptors are thought to reflect the amount of endotoxin—cell interaction over prolonged time intervals. In contrast, endotoxin itself has a short plasma half-life time (in the range of 10 to 30 min). This may explain why sCD14 levels are more closely related to the cytokine levels than endotoxin levels, as shown here and previously [4]. PCT plasma levels have been suggested to be indicative of systemic bacterial infections and are less prominent in endotoxemia [1,6], although the mechanisms are not clear. This study showed only a trend for raised PCT (procalcitonin) levels in oedematous CHF patients (ANOVA: p<0.08), and therefore only low grade bacteraemia, if at all, may be present. That conclusion is supported by results from FACS analysis, showing only moderate changes in the pattern of cellular immune activation. Additionally, the levels of endotoxin observed in this study were well below those otherwise seen in septic shock [19]. The CHF patients studied here had no sign of active infection, and the moderate increase of plasma endotoxin levels is in keeping with the hypothesis of a translocation process. Possibly, it is endotoxin itself rather than bacteria which translocates. Although intensified diuretic therapy resulted in normalisation of endotoxin levels, treatment did not lead immediately to reduced cytokine plasma levels, which is in keeping with a previous study [20]. This may be due to a concentration effect due to the loss of up to 5 kg body water therefore concentrating plasma levels or due to prolonged activation of monocytes/macrophages following exposure to an endotoxin stimulus during a phase of clinical deterioration with increased venous congestion, ie “normalised” endotoxin levels may still cause increased cytokine production. Indeed, such an increased cellular LPS sensitivity has recently been documented for CHF patients with acute decompensation [21], and increased TNF-α releases at baseline and after endotoxin stimulation have recently been found in cardiomyocytes from cardiac transplantation recipients, particularly for those with heart failure of ischaemic aetiology [22]. Also the previously documented raised TNF-α levels in cardiac tissue of end-stage CHF patients [3] may be due to cardiomyocytes or tissue monocytes producing increased amounts of cytokines upon stimulation by LPS, either because these patients were decompensated or because the cardiomyocytes were hypersensitive. After a prolonged phase of clinical stability TNF-α plasma levels showed a strong trend to decrease back to normal, ie the normalisation of the relative cytokine secretion capacity may be a slow process.

Tolerance of monocytes/macrophages to endotoxin can be induced both in vivo and in vitro by endotoxin itself, and for instance it frequently occurs after severe injury [23]. One important mediator of LPS hyposensitivity is IL-10 [24]. Compared to controls, we previously found IL-10 to be lower in stable CHF patients [4]. Glucocorticoids are well known to be able to suppress LPS triggered immune activation [25], and for their general immuno suppressive effects they are considered standard in the treatment of transplant patients. Nevertheless, glucocorticoids are under certain circumstances also a prerequisite for an increased immune response [26]. In CHF patients we have recently shown that the cortisol/DHEA ratio is closely related to the degree of immune activation [27]. This marker of catabolic/anabolic balance is highest in cachectic CHF patients [2], who also demonstrate pronounced immune activation [1,2]. Increased cardiac wall stress and tissue hypoxia (both via local free radical generation and subsequent stimulation of the nuclear factor-kappaB pathway [28]) and hormonal catabolic/anabolic imbalance may cause immunological hypersensitivity, and endotoxin may thus be an important stimulus for cytokine production both in the heart and in the periphery. In vitro already low levels of LPS have detrimental effects on cardiomyocytes [29]. In vivo there may be a dynamic balance between heart function and immune activation in CHF patients [30]. Over time patients with frequent oedematous episodes may suffer most from the cardio-depressant [31,32] and metabolic [33,34] consequences of raised TNF-α levels, arguing for a tight control of the fluid balance of CHF patients.

In stable ambulatory patients Munger et al [35] have not been able to show a significant spill-over of cytokines from the heart, suggesting that cardiac production could not be the main source of the raised peripheral cytokine plasma levels. Supporting the importance of peripheral hypoxia, recently measures of increased oxidative stress have been found to correlate with sTNFR-1/2 levels [36]. We have shown that post-ischaemic peak leg blood flow in clinically stable CHF patients is inversely related to TNF-α plasma levels [37]. This may be due to a relationship between hypoxia and TNF-α production, or alternatively due to toxic effects of TNF-β on endothelial function [38]. Hypoxia per se may not be the most important cytokine trigger in CHF patients because of differences in the cytokine profile. Raised IL-6 plasma levels can be attributed to peripheral hypoxic conditions [39] that will certainly occur in CHF [40], but there is no report that hypoxia per se induces TNF-α, PCT, sTNF-R1 or sTNF-R2 [41]. Increased levels of soluble TNF-α receptors and particularly sCD14 are, in contrast, characteristic of endotoxin action, but not of hypoxic conditions [42].

### CONCLUSION

This study demonstrates the presence of raised plasma endotoxin concentrations in patients with CHF and peripheral oedema. In the presence of unchanged levels of endotoxin binding protein this reflects a potentially pathogenic situation leading to cytokine induction. We show that normalisation of endotoxin levels can be achieved by intensified diuretic treatment. Bacterial endotoxin may be an important stimulus of immune activation in patients with chronic heart failure.

### EXAMPLE 2

### Experimental Trials Relating to the Use of Compounds Able to Bind LPS in Treating Chronic Heart Failure or Acute Heart Failure

Invasive assessments looking for LPS levels in different locations in the body (left and right ventricle, hepatic vein, renal vein, peripheral vein and artery, coronary sinus) may be made in patients with decompensated CHF and myocardial infarction.

This may help in confirming the source of the LPS. If LPS is highest in the hepatic vein this may indicate that the liver or more likely the bowel is the source of LPS. If LPS is higher in the hepatic vein compared to the left ventricle the lung is excluded as a source of LPS.

Gut permeability assessments may be made using sugar absorption tests in patients with and without oedema and control subjects. The precise mechanism of LPS uptake through the bowel is not clear; sugar absorption may reflect this pathway. However, kidney dysfunction (frequent in heart failure) may complicate interpretation of the results.

UDCA may be tested in patients (with oedema or with cardiac cachexia) in comparison with a placebo.

The relationship between LPS plasma levels and prognosis in oedematous and non-oedematous heart failure patients may be investigated.

### EXAMPLE 3

### Lipoproteins and Mortality in Chronic Heart Failure

We explored the relationship of plasma lipoprotein levels in 114 CHF patients (age 63±1 years, New York Heart Association (NYHA) functional class 2.6±0.1, peak VO₂ 17±0.6 ml/kg/min, left ventricular ejection fraction (LVEF) 28±2%, mean±SEM). During mean follow-up of 3 years (>6 months in all patients), 48 patients died (42%). Low cholesterol levels (in mmol/l, all assessed at initial visit in fasting state) predicted impaired 2-year-mortality (hazard ratio (RR) 1.6 per mmol/l reduction, p<0.01), as did low LDL levels (RR 1.5 per mmol/l, p<0.05), triglycerides (RR 2.1 per mmol/l, p<0.01), peak VO₂ (RR 1.3, p<0.0001), NYHA class (RR 3.2, p<0.0001), and age (RR 1.04, p<0.05). Cholesterol levels<5.2 mmol/l (=current guidelines cut-off level above which statin therapy should be started to lower lipoprotein levels in patients with coronary artery disease), below median (i.e. <5.3 mmol/l, both RR>3.2, p<0.01), and in the lowest tertile (<4.8 mmol/l, RR 2.2, p<0.05) were predictive of impaired mortality, independent of heart failure aetiology, albumin levels (i.e. hepatic function), age, peak VO₂, and NYHA class (all p<0.01). Conclusion: Low cholesterol levels independently predict increased mortality in patients with heart failure.

### EXAMPLE 4

### Serum Lipoproteins Inhibit LPS-Activity

LPS-induced cytokine synthesis can be inhibited by serum lipoproteins. However, this is not easily seen experimentally, as it needs a certain pre-incubation procedure (18 hours at 37° C.) that we developed in our laboratory. Only when this protocol is applied it can be seen that normal lipoprotein containing serum exhibits a strong LPS-inhibitory activity, whereas lipoprotein-deficient serum lacks this activity (FIG. 3).

Methodology for example 4, 5, 7, and 8: TNF assessment: ELISA established in the laboratory of Dr. Schumann using 2 monoclonal antibodies (Pharmingen Inc., USA). Recombinant LBP: produced in the lab of Dr. Schumann. Lipoproteins: isolated from sera of healthy young volunteers, isolated by density gradient centrifugation, monocytes isolated also from blood of healthy young volunteers

### EXAMPLE 5

### LDL, HDL, and VLDL Inhibit LPS-Activity when Added to Lipoprotein-Free Serum

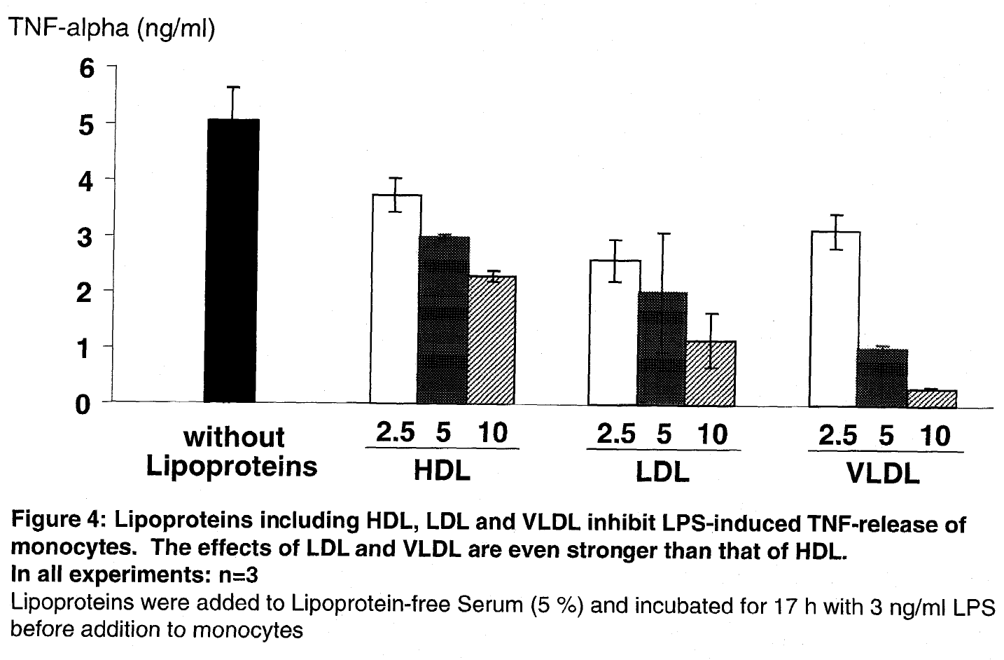

Applying the in vitro system as in example B with monocytes and pre-incubated serum a strong dose-dependent LPS-inhibitory activity of HDL, LDL, and VLDL can be observed (FIG. 4). Again, serum lacking lipoproteins was unable to block LPS-induced TNF synthesis. The ability to block LPS-induced TNF synthesis could be restored by addition of isolated lipoproteins. Additionally this example shows that the effects of LDL and VLDL are even stronger than that of HDL (inhibition of LPS-induced TNF synthesis of LDL and VLDL 40 to 150% stronger than for HDL). Methodology as in example 4

### EXAMPLE 6

### Lipoproteins and Whole Blood Cytokine Production in Chronic Heart Failure

We investigated in whole blood cultures of 18 patients with chronic heart failure and 6 healthy control subjects, tumor necrosis factor-α (TNF) production upon stimulation with LPS and its relationship to the measured plasma HDL levels.

Whole Blood Cultures: Whole blood was anti-coagulated with citrate dextrose (ACD), allowed to rest for 24 hours and then stimulated for 24 h with 100 μg/ml of LPS (Escherichia coli 0111:B4, Sigma, Amersham, U.K.) at 37° C. in 5% CO₂ in 2 ml Eppendorf tubes. Cell-free supernatants, obtained by centrifugation at 12000 rpm were collected and stored in aliquots at −70° C. until analysis.

ELISA assays: Culture supernatants and plasma samples were tested for TNF-α content by commercial sandwich enzyme-linked immunosorbent assays (ELISAs, R&D Systems). ELISAs were performed exactly according to the manufacturer's instructions. Briefly, monoclonal anti-TNF-α antibody was coated (4 μg/ml) onto a microtitre plate (NUNC maxisorp 96 well flat bottomed plates, GIBCO BRL, Paisley, U.K.) to which standards and samples were added. An enzyme-linked polyclonal antibody (300 ng/ml) specific for TNF-α was added to the wells to sandwich-immobilised TNF-α. Addition of a stabilized chromogen and hydrogen peroxide (Pharmingen, San Diego, USA) allowed color development in proportion to the amount of TNF-α. Following a 30-minute incubation period, the assay was stopped by addition of 50 μl/well of 1M Sulphuric acid. TNF-α was assayed by measurement of optical density using a spectrophotometer set to 450 nm (Anthos reader 2001; Anthos Labtec Instrument, Salzburg, Germany). Concentrations were obtained by interpolation on the standard curves using Microsoft Excel. The final concentrations in each sample were calculated as the mean of the results at the proper sample dilution yielding optical densities in the linear parts of the calibration curves. The limit of detection was 16 pg/ml for TNF-α.

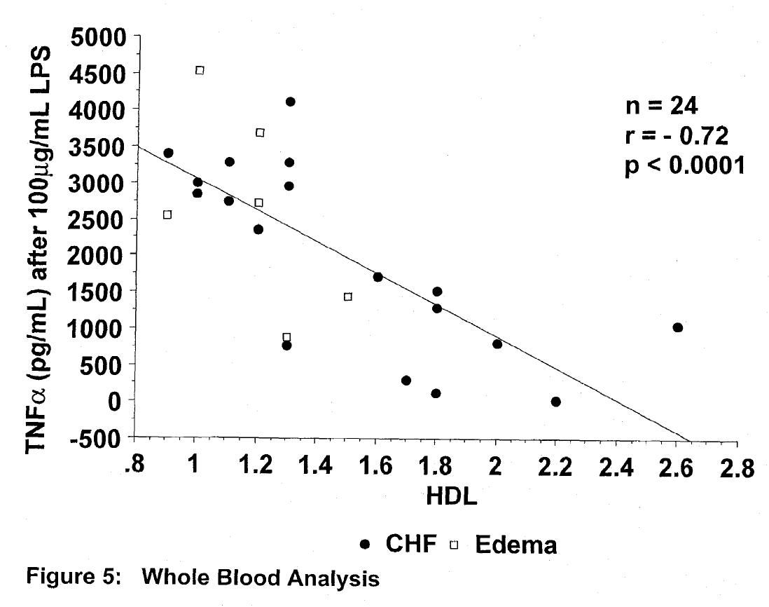

Results: High HDL levels significantly related to low TNF production (r=−0.5 μp<0.05) in 18 CHF patients alone, and in the whole group of 24 subjects (r=−0.72, p<0.0001), see FIG. 5.

Conclusion: High lipoprotein plasma levels relate to lower cytokine production after LPS stimulus.

### EXAMPLE 7

### LBP and Lipoprotein Interaction to Block LPS-Induced TNF Production

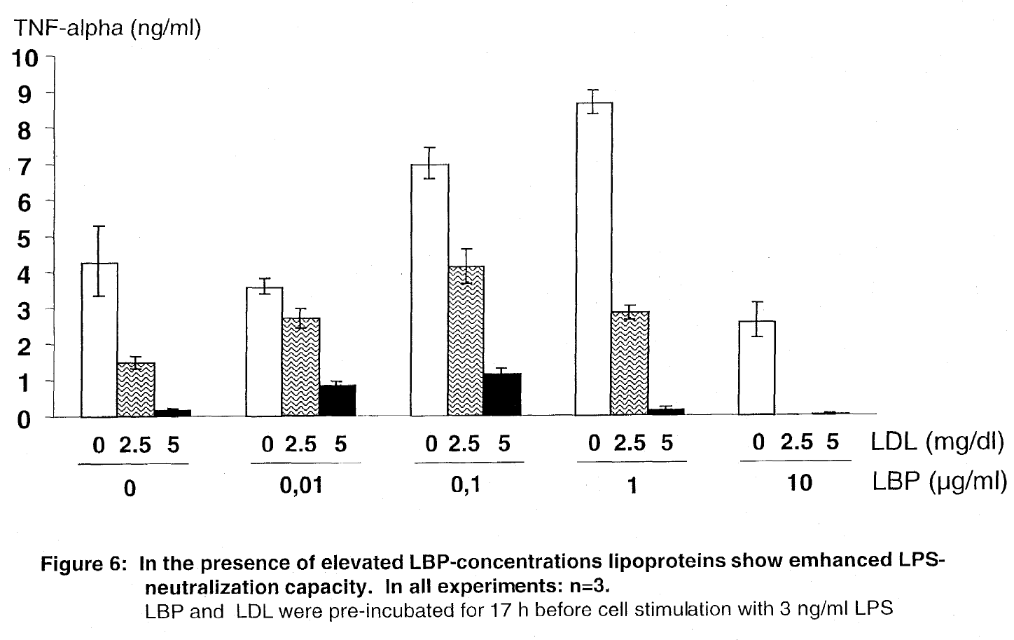

When both, LBP and LDL are titrated into lipoprotein-deficient serum it can be observed that while high levels of LBP inhibit LPS activity, a complete inhibition of LPS activity best can be observed when both LBP and LDL are present (FIG. 6). In additional experiments, we found that principally the same results were obtained using HDL or VLDL instead of LDL. These interactions are novel findings. It is the first time that such high LBP doses could be tested. Methodology as in example 4

### EXAMPLE 8

### LBP can Inhibit LPS-Induced TNF Production in Lipoprotein Containing Serum

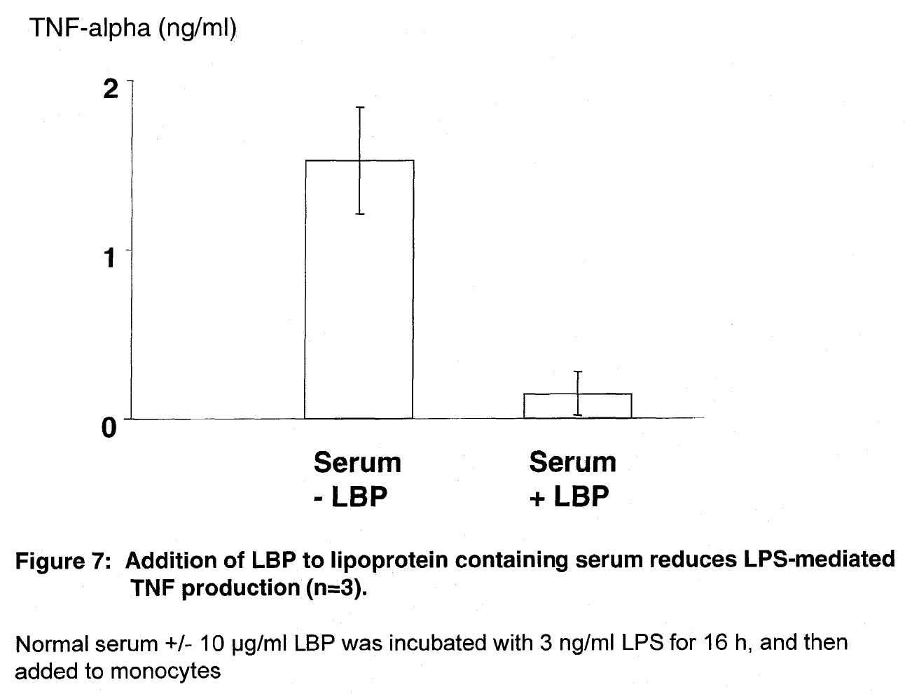

Addition of high concentrations of recombinant human LBP to normal serum (containing lipoproteins) reduces LPS-stimulated TNF production in a monocyte stimulation system (FIG. 7). Methodology as in example 4

### EXAMPLE 9

### LBP in Cardiogenic Shock, i.e. Very Severe Acute Heart Failure

It has been shown previously that LBP enhances LPS effects in serum-free in vitro systems. This, as we have found now, is due to the absence of lipoproteins (FIG. 6). Thus, especially when LBP is elevated and serum lipoproteins are reduced, as it is the case in the diseases described here, it is important to add lipoproteins in order to successfully block endotoxin action. In acute heart failure patients with cardiogenic shock our first LBP-measurements in 10 patients show clearly elevated LBP levels averaging 50.1±27.3 μg/ml (approx. 5-10-fold more than in healthy controls). Furthermore, these patients display generally reduced lipoprotein levels. This situation according to our findings leads to a pro-inflammatory situation that has to be counteracted by addition of lipoproteins, and/or addition of LBP.

### EXAMPLE 10

We have tested the ability of ursodeoxycholic acid (UDCA, FALK Pharma GmbH) to inhibit LPS-mediated TNF production in whole blood of healthy control subjects.

Methods: Heparinized whole blood was diluted 1:10 with medium+/−LPS (50 pg/ml), +/−BPI (1 μg/ml), and +/−UDCA (I 1 g/ml-1 mg/ml) according to the manufactorer's recommendation (Milenia whole blood assay; DPC Biermann, Bad Nauheim, Germany) and incubated for 4 hours at 37° C. In the supernatant, we assessed concentrations of TNF and IL-6 using the semiautomated Immulite system (DPC-Biermann, Bad Nauheim, Germany).

Results: LPS-stimulated cytokine production was inhibited by UDCA independently of the effects of the ethanol solution. 1 mg/ml UDCA reduced LPS-stimulated TNF and IL6 production by >95% in all cases (ethanol 1% alone on average only 32.5% for TNF and 25% for IL6). 100 μg/ml UDCA reduced LPS-stimulated TNF and IL6 production by 68% and 43%, respectively (ethanol 0.1% alone on average only 10% for TNF and 11% for IL6).

Conclusion: This is the first documentation that LPS-stimulated cytokine production of whole blood can be inhibited by application of ursodeoxycholic acid (UDCA).

### EXAMPLE 11

We have tested the ability of ursodeoxycholic acid (UDCA, FALK Pharma GmbH) and BPi to inhibit LPS-mediated TNF production in whole blood of patients with cachexia.

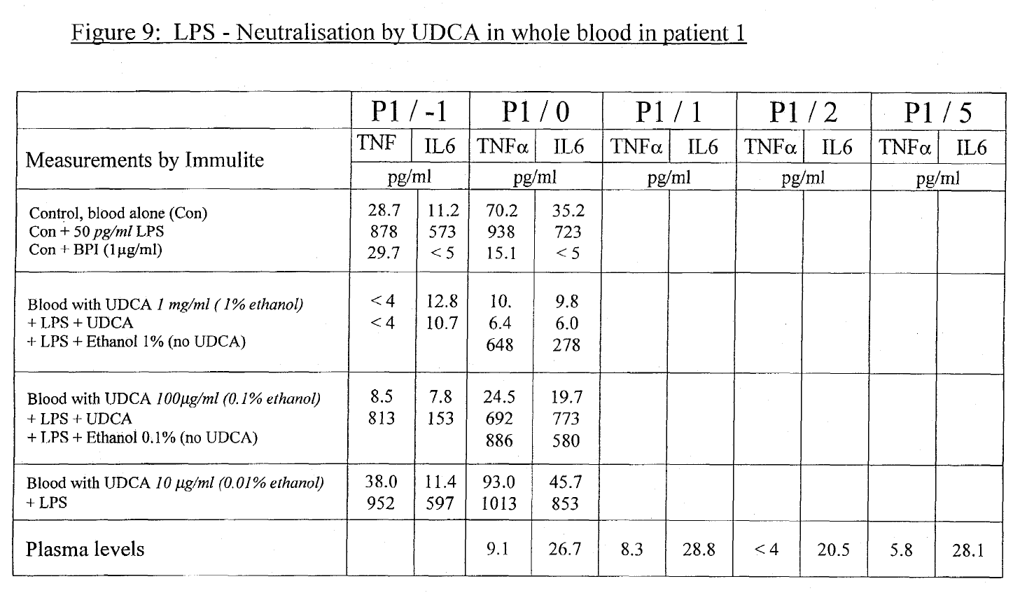

We studied 4 patients with cachexia due to liver cirrhosis. The patients had all weight loss >7.5% compared to their previous normal weight. In 3 of the 4 patients had a alcoholic aetiology. All patients were studied twice on 2 subsequent days (day “−1” and day “0”), see FIG. 9 to 12.

Methods: Heparinized whole blood was diluted 1:10 with medium+/−LPS (50 pg/ml), +/−BPI (1 μg/ml), and +/−UDCA (1 μg/ml-1 mg/ml) according to the manufactorer's recommendation (Milenia whole blood assay; DPC Biermann, Bad Nauheim, Germany) and incubated for 4 hours at 37° C. In the supernatant, we assessed concentrations of TNF and IL-6 using the semiautomated Immulite system (DPC-Biermann, Bad Nauheim, Germany).

Results: In patients with cachexia due to liver cirrhosis spontaneous (“Control” data) and LPS-stimulated production of TNF and IL6 is significantly elevated compared to that of healthy subjects. LPS-stimulated cytokine production was inhibited by UDCA independently of the effects of the ethanol solution. The detailed results are presented in FIG. 9 to 12. 1 mg/ml UDCA reduced LPS-stimulated TNF production on average by >99% and IL6 production by 97% (ethanol 1% alone on average only by 38% for TNF and 43% for IL6). 100 μg/ml UDCA reduced LPS-stimulated TNF and IL6 production by 42% and 13%, respectively, ethanol 0.1% alone on average only 9% for TNF and IL6 production increased by 18% for ethanol alone).

BPi (1 μg/ml) reduced significantly the spontaneous production of TNF and IL6 of whole blood of patients with cachexia due to liver cirrhosis. In 8 experiments 6 times TNF and IL6 levels, respectively, were lowered by at least 5 pg/ml or towards non-detectability, and only in 2 cases TNF and IL6 levels remained stable (p<0.05 for changes).

Conclusion: This is the first documentation that LPS-stimulated cytokine production of whole blood of patients with cachexia can be inhibited by in vitro application of ursodeoxycholic acid (UDCA). This is the first documentation that spontaneous production of inflammatory cytokines in whole blood of patients with cachexia can be inhibited by application of BPi in vitro.

### EXAMPLE 12

We have tested the ability of the therapeutic application of ursodeoxycholic acid (UDCA, FALK Pharma GmbH) to lower plasma levels of TNF and IL6 and to lower spontaneous and LPS-stimulated whole blood cytokine production in patients with cachexia.

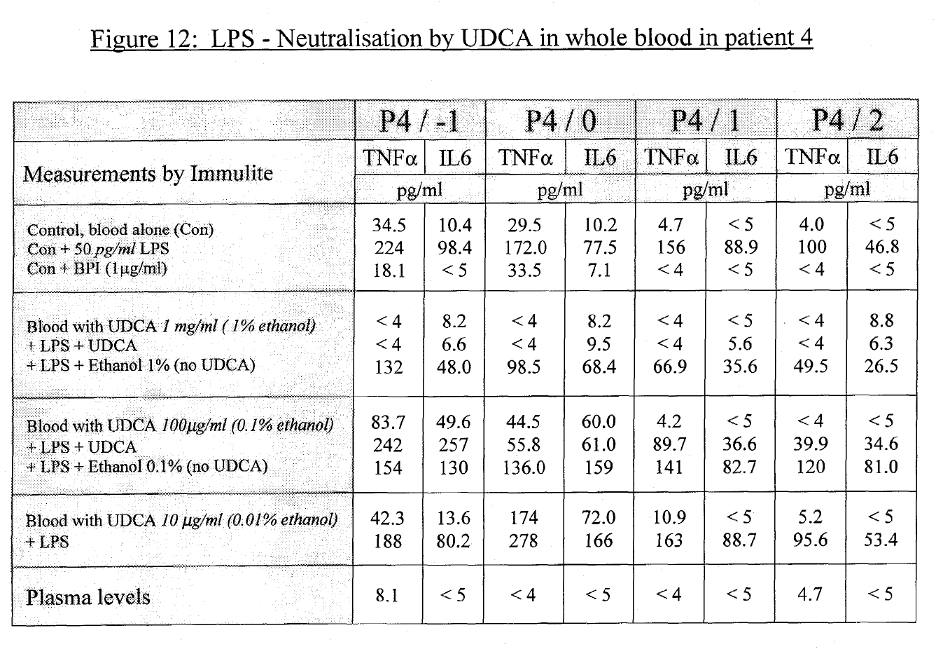

We studied in 2 patients with cachexia due to liver cirrhosis plasma cytokine levels after treatment with 3 times 250 mg daily UDCA (FALK Pharma GmbH). The patients had weight loss >7.5% compared to their previous normal weight. The patients were studied at baseline prior to the treatment on 2 subsequent days (day “−1” and day “0”), and then they were restudied on day 1 (“1”), day 2 (“2”), and day 5 (“5”), see FIGS. 9 and 12.

Methods: Heparinized whole blood was diluted 1:10 with medium+/−LPS (50 pg/ml), +/BPI (1 μg/ml), and +/−UDCA (1 μg/ml-1 mg/ml) according to the manufactorer's recommendation (Milenia whole blood assay; DPC Biermann, Bad Nauheim, Germany) and incubated for 4 hours at 37° C. In the supernatant and in plasma, we assessed concentrations of TNF and IL-6 using the semiautomated Immulite system (DPC-Biermann, Bad Nauheim, Germany).

Results: Only patient 1 showed elevated plasma levels at baseline (FIG. 9). During 5 days of treatment plasma levels of TNF were lower. In patient 4 we were able to reassess whole blood TNF and IL6 production after 1 and 2 days of treatment with UDCA. Spontaneous production of TNF and IL6 in whole blood was reduced substantially to almost undetectable levels. After 2 days of UDCA treatment LPS-stimulated cytokine production was found to be lowered by 43.5% for TNF and by 39.6% for IL6.

Conclusion; This is the first documentation that LPS-stimulated cytokine production of whole blood of patients with cachexia can be inhibited by in vivo therapeutic application of ursodeoxycholic acid (UDCA). This is the first documentation that plasma levels of TNF alpha of patients with cachexia can be inhibited by application of BPi.

### EXAMPLE 13

### Endotoxin in Cachectic Patients with Liver Cirrhosis

It has never been studied, whether endotoxin (LPS) or a marker of endotoxaemia may be raised in patients with liver cirrhosis who suffer from cachexia. Plasma levels of soluble CD14 (sCD14) can reflect the history of LPS-cell interaction (Anker et al., Am J Cardiol 1997; 79:1426-1430.).

We investigated in 46 patients with liver cirrhosis (54±12 years, female 15, male 31, Child A:B:C=24:13:9), alcoholic aetiology in 32 patients) resting energy expenditure (REE, indirect calorimetry), food intake diaries, fat mass (skin fold thickness and calculation according to standard formulae) and body cell mass (BCM, body impedance, Data Input 2000, USA). Soluble CD14 was measured by ELISA (R&D Systems). The majority of patients had a BCM of <35% of body weight (mean±standard deviation: 25±7%, median 33%, range 11.8-41.9%). Plasma sCD14 levels were significantly increased in patients (mean±standard deviation: 4045±623 pg/ml, median 3920 pg/ml, range 2960-5460 pg/ml) compared to sCD14 levels of healthy individuals (mean: 2714 pg/ml, upper limit of normal 3711 pg/ml, as published in Anker et al., Am J Cardiol 1997; 79:1426-1430).

The patients with low BCM relative to their body weight must be considered to suffer from wasting disease, which was the majority in this study (63% of patients had a BCM <35%/kg body weight). The majority of patients in this study were metabolically catabolic as evidenced by a REE/BCM coefficient of 67±19 kcal/kg BCM (range 43-163, normal range in healthy subjects: 45-55 kcal/kg).

The strongest correlation that we found was between the degree of wasting (BCM per kg body weight) and the marker of endotoxaemia, i.e. soluble CD14 (r=−0.565, p<0.001). This means, the lower the relative BCM (i.e. the more cachectic) a patient was the higher the were also the sCD14 plasma levels. Plasma levels of sCD14 also correlated closely and directly with the degree of catabolic energetic/metabolic status (i.e. the REE/BCM coefficient), r=0.549, p<0.001.

Conclusion: This is the first study suggesting that endotoxin (LPS) levels in patients with liver cirrhosis may be particularly high in patients with cachexia. This study also suggests that endotoxin (LPS) is causally related to the characteristics of the cachexia syndrome in liver cirrhosis, i.e. reductions in muscle tissue and increases in metabolic rate.

### EXAMPLE 14

### LBP in Cachectic Patients Due to Liver Cirrhosis

We have studied LBP plasma levels in 6 patients with cachexia due to liver cirrhosis. The patients had weight loss >7.5% compared to their previous normal weight. The disease aetiology was thought to be alcoholic in 4 cases and non-alcoholic in 2 cases. In non of these patients increased LBP levels were found (all below 20 μg/ml). High levels LBP can (together with lipoproteins) block LPS mediated production of inflammatory cytokines. We conclude that LBP is lacking in patients with cachexia due to liver cirrhosis, and that the application of LBP, possibly together with lipoproteins, could counteract the inflammatory status seen in these patients.

### REFERENCES

1. Levine B, Kalman J, Mayer L, Fillit H M, Packer M. Elevated circulating levels of tumor necrosis factor in severe chronic heart failure. N Engl J Med 1990; 323:236-241.
2. Anker S D, Swan J W, Chua T P, Ponikowski P, Harrington D, Kox W J, Poole-Wilson P A, Coats A J S. Hormonal changes and catabolic/anabolic imbalance in chronic heart failure: The importance for cardiac cachexia. Circulation 1997; 96:526-534.
3. Torre-Amione G, Kapadia S, Lee J, Durand J-B, Bies R^{D}, Young J B, Mann D L. Tumor necrosis factor-α and tumor necrosis factor receptors in the failing human heart. Circulation 1996; 93:704-711.
4. Anker S D, Egerer K, Volk H-D, Kox W J, Poole-Wilson P A, Coats A J S. Elevated soluble CD14 receptors and altert cytokines in chronic heart failure. Am J Cardiol 1997; 79:1426-1430.
5. Schumann R R, Leong S R, Flaggs G W, Gray P W, Wright S D, Mathison J C, Tobias P S, Ulevitch R J. Structure and function of lipopolysaccharide binding protein. Science 1990; 249, 1429-1431.
6. Tobias, P S, Soldau K, lovinc N M, Elsbach P, Weiss P. Lipopolysaccharide (LPS) binding proteins BPI and LBP form different types of complexes with LPS. J Biol Chem 1997; 272: 18682-18685.
7. Schumann R R, Kirschning C, Unbehaun A, Aberle H, Knopf H-P, Ulevitch R J, Herrmann, F. Lipopolysaccharide binding protein (LBP) is a secretory class 1 acute phase protein requiring binding of the transcription factor STAT-3, C/EBPβ, and AP-1. Mol Cell Biol 1996; 16:3490-3503.
8. Lamping N, Dettmer R, Schröder N W J, Pfeil D, Hallatschek W, Burger R, Schumann R R. LPS-binding protein protects mice from septic shock caused by LPS or gram-negative bacteria. J Clin Invest 1998; 101:2065-2071.
9. Ziegler-Heitbrock, Ulevitch R J. CD14: Cell surface receptor and differentiation marker. Immunology Today 1993; 14:121-125.
10. Landmann R, Link S, Sausano S, Rajacic Z, Zimmerli W. Soluble CD14 activates monocytic cells independently of lipopolysaccharide. Infect Immun 1998; 66:2264-2271.
11. Clark A L, Poole-Wilson P A, Coats A J. Exercise limitation in chronic heart failure: central role of the periphery. J Am Coll Cardiol 1996; 28:1092-1102.
12. Anker S D, Coats A J S. Metabolic, functional, and haemodynamic staging for CHF? Lancet 1996; 348:1530-1531.
13. Sautner T, Wessely C, Riegler M, Sedivy R, Gotzinger P, Losert U, Roth E, Jakesz R, Fugger R. Early effects of catecholamine therapy on mucosal integrity, intestinal blood flow, and oxygen metabolism in porcine endotoxin shock. Ann Surg 1998; 228:239-248.
14. Lamping N, Hoess A, Yu B, Park T C, Kirschning C, Pfeil D, Reuter D, Wright S D, Herrmann F, Schumann R R. Effect of site-directed mutagenisis of basic residues (Arg 94, Lys 95, Lys 99) of lipopolysaccharide (LPS)-binding protein on binding and transfer of LPS and subsequent immune cell activation. J Immunol 1996; 157:4648-4656.
15. Dandona P, Nix D, Wilson M F, Aljada A, Love J, Assicot M, Bohoun C. Procalcitonin increase after endotoxin injection in normal subjects. J Clin Endocrinol Metab 1994; 79:1605-1608.
16. Assicot M, Gendrel D, Carsin H, Raymond J, Guilbaud J, Bohuon C. High serum procalcitonin concentrations in patients with sepsis and infection. Lancet 1993; 341:515-518.
17. Wright S D. Multiple receptors for endotoxin. Curr Opin Immunol 1991; 3:83-90.
18. Ulevitch R J, Tobias P S. Receptor-dependent mechanisms of cell stimulation by bacterial endotoxin. Annu Rev Immunol 1995; 13:437-457.
19. Gomez-Jimenez J. Salgado A, Mourelle M, Martin M C, Segura R M, Peracaula R, Moncada S. L-arginine: nitric oxide pathway in endotoxemia and human septic shock. Crit. Care Med 1995; 23:253-258.
20. Vanderheyden M, Kersschot E, Paulus W J. Pro-inflammatory cytokines and endothelium-dependent vasodilation in the forearm. Serial assessment in patients with congestive heart failure. Eur Heart J 1998; 19:747-752.
21. Vonhof S, Brost B, Stille-Siegener M, Grumbach 1M, Kreuzer H, Figulla H R. Monocyte activation in congestive heart failure due to coronary artery disease and idiopathic dilated cardiomyopathy. Int J Cardiol 1998; 63:237-244.
22. Wagner D R, McTiernan C, Sanders V J, Feldman A M. Adenosine inhibits lipopolysaccharide-induced secretion of tumor necrosis factor-alpha in the failing human heart. Circulation 1998; 97:521-524.
23. Keel M, Schregenberger N, Steckholzer U, Ungethum U, Kenney J, Trentz O, Ertel W. Endotoxin tolerance after severe injury and its regulatory mechanisms. J Trauma 1996; 41:430-437.
24. Randow F, Syrbe U, Meisel C, Krausch D, Zuckermann H, Platzer C, Volk H D. Mechanism of endotoxin desensitization: involvement of interleukin 10 and transforming growth factor beta. J Exp Med 1995; 181:1887-1892.
25. Garvy B A, Fraker P J. Suppression of the antigenic response of murine bone marrow B cells by physiological concentrations of glucocorticoids. Immunology 1991; 74:519-523.
26. Wilckens T. Glucocorticoids and immune function: physiological relevance and pathogenic potential of hormonal dysfunction. Trends Pharmacol Sci 1995; 16:193-197.
27. Anker S D, Clark A L, Kemp M, Salsbury C, Teixeira M M, Hellewell P G, Coats A J S. Tumor necrosis factor and steroid metabolism in chronic heart failure: possible relation to muscle wasting. J Am Coll Cardiol 1997; 30:997-1001.
28. Barnes P J, Karin M. Nuclear factor-kappaB: a pivotal transcription factor in chronic inflammatory diseases. N Engl J Med 1997; 336:1066-1071.
29. Lew W Y, Ryan J, Yasuda S. Lipopolysaccharide induces cell shrinkage in rabbit ventricular cardiac myocytes. Am J Physiol 1997; 272:H2989-H2993.
30. Bachetti T, Ferrari R. The dynamic balance between heart function and immune activation. Europ Heart J 1998; 19:681-682.
31. Kelly R A, Smith T W. Cytokines and cardiac contractile function. Circulation 1997; 95:778-781.
32. Torre-Amione G; Kapadia S; Lee J; Bies R D; Lebovitz R; Mann D L. Expression and functional significance of tumor necrosis factor receptors in human myocardium. Circulation 1995; 92: 1487-1493.
33. Tracey K J, Morgello S, Koplin B, Fahey T J III, Fox J, Aledo A, Manogue K R, Cerami A. Metabolic effects of cachectin/tumor necrosis factor are modified by site of production: Cachectin/tumor necrosis factor-secreting tumor in skeletal muscle induces chronic cachexia, while implantation in brain induces predominantly acute anorexia. J Clin Invest 1990; 86:2014-2024.
34. Bristow M R. Tumor necrosis factor- and cardiomyopathy. Circulation 1998; 97:1340-1341.
35. Munger M A, Johson B, Amber I J, Callahan K S, Gilbert E M. Circulating concentrations of proinflammatory cytokines in mild or moderate heart failure secondary to ischemic or ideopathic dilated cardiomyopathy. Am J Cardiol 1996; 77:723-727.
36. Keith M, Geranmayegan A, Sole M J, Kurian R, Robinson A, Omran A S, Jeejeebhoy K N. Increased Oxidative Stress in Patients With Congestive Heart Failure. J Am Coll Cardiol 1998; 31:1352-1356.
37. Anker S D, Volterrani M, Egerer K R, Felton C V, Kox W J, Poole-Wilson P A, Coats A J S. Tumor necrosis factor alpha as a predictor of peak leg blood flow in patients with chronic heart failure. Q J Med 1998; 91:199-203.
38. Tracey K J, Cerami A. Tumor necrosis factor, other cytokines and disease. Ann Rev Cell Biol 1994; 10:317-43.
39. Yan S F, Tritto I, Pinsky D, Liao H, Huang J, Fuller G, Brett J, May L, Stem D. Induction of interleukin-6 (IL-6) by hypoxia in vascular cells. J Biol Chem 1995; 270: 11463-11471.
40. Munger M A, Stanek E J, Nara A R, Strohl K P, Decker M J, Nair R N. Arterial oxygen saturation in chronic congestive heart failure. Am J Cardiol 1994; 73:180-185.
41. Klein C L, Kohler H, Bittinger F, Otto M, Hermanns I, Kirkpatrick C J. Comperative studies on vascular endothelium in vitro. 2.Hypoxia: its influences on endothelial cell proliferation and expression of cell adhesion molecules. Pathobiology 1995; 63:1-8.
42. Eggesbo J B, Hjernann I, Lund P K, Joo G B, Ovstebo R, Kierulf P. LPS-induced release of IL-1 beta, IL-6, IL-8, TNF-alpha and sCD14 in whole blood and PBMC from persons with high or low levels of HDL-lipoprotein. Cytokine 1994; 6:521-529.

TABLE 1
Characteristics of chronic heart failure (CHF) patients with
and without peripheral edema compared to healthy volunteers.

| | healthy volunteers | CHF - no edema | CHF - edema | p (ANOVA) |
|---|---|---|---|---|
| n | 14 | 20 | 20 | |
| age | 55 ± 4 | 63 ± 4 | 64 ± 2 | |
| NYHA class | | 2.6 ± 0.2 | 3.3 ± 0.1 ### | |
| weight [kg] | 74 ± 7 | 76 ± 7 | 78 ± 8 | |
| etiology: ischemic | | 16 | 11 | |
| idiopathic dilative | | 4 | 9 | |
| sodium [mmol/L] | 139 ± 0.4 | 137 ± 1.2 | 134 ± 1.1 ** | <0.006 |
| creatinine [μmol/L] | 82 ± 4 | 131 ± 14 | 219 ± 37 *** # | <0.003 |
| urea [mmol/L] | 5.4 ± 0.2 | 11.0 ± 2.0 | 20.0 ± 2.9 *** ## | <0.0003 |
| uric acid [μmol/L] | 308 ± 17 | 417 ± 42 * | 640 ± 53 *** ### | <0.0001 |
| ASAT [IU/L] | 26 ± 3 | 24 ± 2 | 23 ± 2 | |
| ALAT [IU/L] | 23 ± 3 | 17 ± 1 * | 14 ± 1 ## | <0.01 |

Legend:
* p < 0.05,
** p < 0.01,
*** p < 0.001 vs healthy volunteers;
#: p < 0.05,
##: p < 0.01,
###: p < 0.001 vs no edema;
NYHA—New York Heart Association;
ASAT—aspartate aminotransferase;
ALAT—alanine aminotransferase

TABLE 2
Plasma levels of endotoxin and inflammatory markers in healthy
volunteers and patients with chronic heart failure (CHF).

| | healthy volunteers | CHF - no edema | CHF - edema | p (ANOVA) |
|---|---|---|---|---|
| endotoxin [IU/mL] | 0.46 ± 0.05 | 0.37 ± 0.05 | 0.74 ± 0.10 * ### | <0.003 |
| TNF-α [pg/mL] | 24.6 ± 2.4 | 25.8 ± 1.8 | 36.6 ± 2.8 ** ## | <0.001 |
| sTNF-R1 [pg/mL] | 708 ± 57 | 1077 ± 118 | 1922 ± 313 *** ## | <0.001 |
| sTNF-R2 [pg/mL] | 1465 ± 264 | 2096 ± 330 | 3143 ± 388 ** # | <0.01 |
| sCD14 [ng/mL] | 3456 ± 156 | 3674 ± 102 | 4243 ± 154 *** ## | <0.001 |
| procalcitonin [ng/ml] | 87 ± 4 | 106 ± 16 | 145 ± 21 | =0.073 |
| interleukin-6 [pg/mL] | 2.0 ± 0.1 | 4.3 ± 1.2 | 14.7 ± 3.9 ** ## | <0.003 |
| CRP [mg/L] | 5.6 ± 0.5 | 9.5 ± 1.6 | 19.7 ± 4.6 ** # | <0.003 |

Legend:
* p < 0.05,
** p < 0.01,
*** p < 0.001 vs healthy volunteers;
#: p < 0.05,
##: p < 0.01,
###: p < 0.001 vs no edema;
TNF—tumor necrosis factor;
sTNFR—soluble TNF receptor;
sCD14—soluble CD14;
CRP—c-reactive protein

1-75. (canceled)

76. A method for ameliorating or treating endotoxin-mediated TNF-α production in acute or chronic heart failure in a human patient, the method comprising the steps of measuring the level of TNF-α, endotoxin or soluble CD14 in the blood of a human patient and if any such level is elevated, administering to the patient a therapeutically effective amount of a ursodeoxycholic acid, or ursodeoxycholic acid in combination with diuretics.

77. The method according to claim 76, wherein the heart failure is an acute or chronic congestive heart failure with evidence of peripheral edema; the heart failure is severe according to NYHA class III or IV; the heart failure involves a history of decompensation phases; or the heard failure is accompanied by cardiac cachexia.

78. The method according to claim 76 wherein the ursodeoxycholic acid is able to inhibit the TNF-α and IL-6 production in the patients in response to endotoxin.

79. The method according to claim 76 wherein the ursodeoxycholic acid is able to reduce the permeability of the gut wall to bacteria and/or endotoxin.

80. The method according to claim 76 wherein the ursodeoxycholic acid is administered orally.

81. The method according to claim 76 wherein the ursodeoxycholic acid is administered intravenously.

82. The method according to claim 76 wherein the ursodeoxycholic acid is administered rectally.

83. A pharmaceutical formulation comprising ursodeoxycholic acid and a diuretic.
